(19) United States
(12) Patent Application Publication
(10) Pub. No.: US 2021/0077860 A1
(43) Pub. Date: Mar. 18, 2021

(54) REACTIVE PROTOCOLS FOR ORTHOPEDIC TREATMENT

(71) Applicant: ROM TECHNOLOGIES, INC., Las Vegas, NV (US)

(72) Inventors: Daniel Posnack, Fort Lauderdale, FL (US); Peter Arn, Roxbury, CT (US); Wendy Para, Las Vegas, NV (US); S. Adam Hacking, Nashua, NH (US); Micheal Mueller, Oil City, PA (US); Joe Guaneri, Merrick, NY (US); Jonathan Greene, Denver, CO (US)

(73) Assignee: ROM TECHNOLOGIES, INC., Las Vegas, NV (US)

(21) Appl. No.: 17/021,889

(22) Filed: Sep. 15, 2020

Related U.S. Application Data
(60) Provisional application No. 62/901,666, filed on Sep. 17, 2019.

(51) Int. Cl. A63B 24/00; A63B 22/06; A63B 71/06; A63B 21/00; G16H 10/60; G16H 20/30

(52) U.S. Cl. CPC A63B 24/0075; A63B 22/0694; A63B 24/0062; A63B 2022/0094; A63B 21/00178; G16H 10/60; G16H 20/30; A63B 71/0622

(57) ABSTRACT

A computer-implemented system for managing a treatment plan includes a clinician interface to configure various aspects of a treatment plan. The treatment plan includes one or more treatment protocols, and each treatment protocol includes one or more sessions. Each session comprises several session periods, with each of the session periods including a particular activity for treating the body part of the patient. The clinician interface includes a patient profile display presenting historical data regarding performance of the treatment plan, and a protocol management display presenting controls for modifying a treatment protocol within the treatment plan. A treatment apparatus, such as a stationary bicycle, is used by the patient to perform the treatment protocol. A patient interface, such as a tablet computer, communicates information to the patient and receives feedback from the patient. A server stores data and coordinates operation of the system.

### CROSS-REFERENCES TO RELATED APPLICATIONS

This application claims priority to and the benefit of U.S. Provisional Application Patent Ser. No. 62/901,666 filed Sep. 17, 2019, titled “Reactive Protocols for Orthopedic Treatment,” the entire disclosure of which is hereby incorporated by reference for all purposes.

### BACKGROUND

Treatment plans for treatment of a patient may include a plurality of sessions, with each session comprising several session periods, and with each session period including a particular activity for treating a body part of the patient. Such treatment plans may include orthopedic rehabilitation of a bone, muscle group and/or a joint.

### SUMMARY

A computer implemented system for managing a treatment plan is provided. The computer implemented system comprises a clinician interface comprising a patient profile display and a protocol management display, the patient profile display presenting historical data regarding performance of the treatment plan by a patient, and the protocol management display presenting controls for modifying a treatment protocol within the treatment plan. The treatment protocol comprises a plurality of sessions for treatment of a body part of the patient. Each session within the plurality of sessions comprises an ordered combination of session periods, with each session period including a particular activity for treating the body part of the patient. Each session period is selected from a group comprising a passive period, an assisted period, an active period, and a resistance period. The particular activity of each passive period includes moving the body part by an external force. The particular activity of each assisted period includes moving the body part by the patient with assistance of the external force. The particular activity of each active period includes the patient moving the body part without assistance of the external force. The particular activity of each resistance period includes the patient actively moving the body part against a resistance force.

A system for remote treatment is also provided. The system for remote treatment comprises a clinician interface presenting a protocol management display presenting controls for modifying a treatment plan for treatment of a body part of a patient; a patient interface including an output device and an input device for communicating information to and from the patient; and a treatment apparatus configured to be manipulated by the patient for performing a rehabilitation regimen upon the body part, with the body part comprising at least one of a joint, a bone, or a muscle group. The patient interface and the treatment apparatus are each configured to operate from a patient location geographically separate from a location of the clinician interface.

A clinician user interface generated by a computer is also provided. The clinician user interface includes a profile display presenting information regarding a treatment history of a patient. The clinician user interface also includes a protocol management display presenting a treatment plan comprising a plurality of treatment protocols. The clinician user interface also includes a plan modification control configured to modify the plurality of treatment protocols of the treatment plan. Each treatment protocol of the plurality of treatment protocols comprises a plurality of sessions for treatment of a body part of the patient; and each session of the plurality of sessions comprises an ordered combination of session periods, with each of the session periods including a particular activity for treating the body part of the patient.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 shows a block diagram of an embodiment of a computer implemented system for managing a treatment plan;

FIG. 2 shows a perspective view of an embodiment of a treatment apparatus;

FIG. 3 shows a perspective view of a pedal of the treatment apparatus of FIG. 2;

FIG. 4 shows a perspective view of a person using the treatment apparatus of FIG. 2;

FIG. 5 shows an example embodiment of an overview display of a clinician interface;

FIG. 6 shows an example embodiment of a patient profile display of a clinician interface;

FIG. 7 shows another view of the example patient profile display of FIG. 6;

FIG. 8 shows an example embodiment of a treatment plan display of a clinician interface;

FIG. 9 shows an example embodiment of a treatment plan modification display of a clinician interface;

FIG. 10 shows an example embodiment of a treatment protocol management display of a clinician interface;

FIG. 11 shows an example embodiment of a login screen of a patient interface;

FIG. 12 shows an example embodiment of a body part confirmation screen of a patient interface;

FIG. 13 shows an example embodiment of a treatment plan overview screen of a patient interface;

FIG. 14 shows an example embodiment of a pain level input screen of a patient interface;

FIG. 15 shows an example embodiment of a first patient questionnaire screen of a patient interface;

FIG. 16 shows an example embodiment of a second patient questionnaire screen of a patient interface;

FIG. 17 shows an example embodiment of a question confirmation screen of a patient interface;

FIG. 18 shows an example embodiment of a third patient questionnaire screen of a patient interface;

FIG. 19 shows an example embodiment of a positioning confirmation screen of a patient interface;

FIG. 20 shows an example embodiment of a positioning help screen of a patient interface;

FIG. 21 shows an example embodiment of a session period introduction screen of a patient interface;

FIG. 22 shows an example embodiment of an E-stop confirmation screen of a patient interface;

FIG. 23 shows an example embodiment of an adjustment introduction screen of a patient interface;

FIG. 24 shows an example embodiment of an adjustment confirmation screen of a patient interface;

FIG. 25 shows an example embodiment of a session period action screen of a patient interface;

FIG. 26 shows an example embodiment of an exercise introduction screen of a patient interface;

FIG. 27 shows an example embodiment of an exercise action screen of a patient interface;

FIG. 28 shows an example embodiment of a first progress data screen of a patient interface; and

FIG. 29 shows an example embodiment of a first progress data screen of a patient interface.

### NOTATION AND NOMENCLATURE

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

The terminology used herein is for the purpose of describing particular example embodiments only, and is not intended to be limiting. As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer, or section from another region, layer, or section. Terms such as “first,” “second,” and other numerical terms, when used herein, do not imply a sequence or order unless clearly indicated by the context. Thus, a first element, component, region, layer, or section discussed below could be termed a second element, component, region, layer, or section without departing from the teachings of the example embodiments. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Spatially relative terms, such as “inner,” “outer,” “beneath,” “below,” “lower,” “above,” “upper,” “top,” “bottom,” and the like, may be used herein. These spatially relative terms can be used for ease of description to describe one element's or feature's relationship to another element(s) or feature(s) as illustrated in the figures. The spatially relative terms may also be intended to encompass different orientations of the device in use, or operation, in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptions used herein interpreted accordingly.

Moreover, various functions described below can be implemented or supported by one or more computer programs, each of which is formed from computer readable program code and embodied in a computer readable storage medium. The terms “application” and “program” refer to one or more computer programs, software components, sets of instructions, procedures, functions, methods, objects, classes, instances, related data, or a portion thereof adapted for implementation in a suitable computer readable program code. The phrase “computer readable program code” includes any type of computer code, including source code, object code, and executable code. The phrase “computer readable storage medium” includes any type of medium capable of being accessed by a computer, such as read only memory (ROM), random access memory (RAM), a hard disk drive, a flash drive, a compact disc (CD), a digital video disc (DVD), solid state drive (SSD), or any other type of memory. A “non-transitory” computer readable storage medium excludes wired, wireless, optical, or other communication links that transport transitory electrical or other signals. A non-transitory computer-readable storage medium includes media where data can be permanently stored and media where data can be stored and later overwritten, such as a rewritable optical disc or an erasable memory device.

The terms “transmit,” “receive,” and “communicate,” as well as derivatives thereof, encompass both direct and indirect communication. The terms “transmit,” “receive,” and “communicate,” as well as derivatives thereof, encompass both communication with remote systems and communication within a system, including reading and writing to different portions of a memory device. The term “controller” means any device, system or part thereof that controls at least one operation. Such a controller may be implemented in hardware or a combination of hardware and software and/or firmware. The functionality associated with any particular controller may be centralized or distributed, whether locally or remotely.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

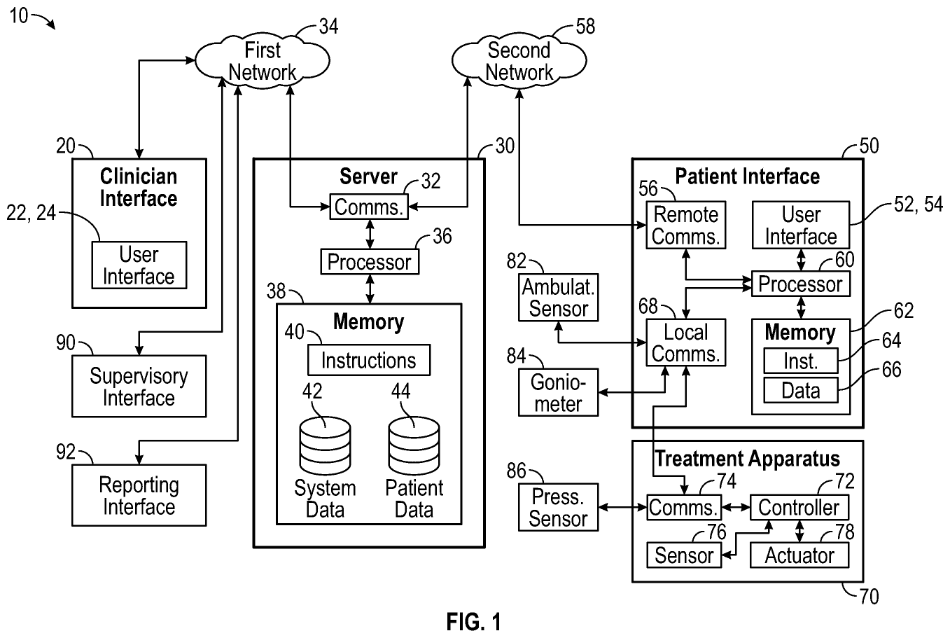

FIG. 1 shows a block diagram of a computer-implemented system 10, hereinafter called “the system” for managing a treatment plan. The treatment plan includes one or more treatment protocols, and each treatment protocol includes one or more sessions. Each session comprises several session periods, with each session period including a particular activity for treating the body part of the patient. For example, a treatment plan for post-operative rehabilitation after a knee surgery may include an initial treatment protocol with twice daily stretching sessions for the first 3 days after surgery and a more intensive treatment protocol with active exercise sessions performed 4 times per day starting 4 days after surgery.

The system 10 includes a clinician interface 20 for a clinician, such as a doctor, a nurse, a physical therapist, or a technician, to use to review and to configure various aspects of a treatment plan for use in treating a patient. The clinician interface 20 includes a clinician input device 22 and a clinician display 24, which may be collectively called a clinician user interface 22, 24. The clinician input device 22 may include one or more of a keyboard, a mouse, a trackpad, or a touch screen, for example. Alternatively or additionally, the clinician input device 22 may include one or more microphones and voice-based functionalities, with hardware and/or software configured to interpret spoken instructions by the clinician by using the one or more microphones. The clinician input device 22 may include functionality provided by or similar to existing voice-based assistants such as Siri by Apple, Alexa by Amazon, Google Assistant, or Bixby by Samsung. The clinician input device 22 may include other hardware and/or software components. The clinician input device 22 may include one or more general purpose devices and/or special-purpose devices.

The clinician display 24 may take one or more different forms including, for example, a computer monitor or display screen on a tablet, smartphone, or a smart watch. The clinician display 24 may include other hardware and/or software components such as a projector, virtual reality capability, or augmented reality capability etc. The clinician display 24 may incorporate various different visual, audio, or other presentation technologies. For example, the clinician display 24 may include a non-visual display, such as an audio signal, which may include spoken language and/or other sounds such as tones, chimes, and/or melodies which may signal different conditions and/or directions. The clinician display 24 may comprise one or more different display screens presenting various data and/or interfaces or controls for use by the clinician. The clinician display 24 may include graphics, which may be presented by a web-based interface and/or by a computer program or application (App.).

The system 10 also includes a server 30 configured to store and to provide data related to managing the treatment plan. The server 30 may include one or more computers and may take the form of a distributed and/or virtualized computer or computers. In some embodiments, the server 30 may generate aspects of the clinician display 24 for presentation by the clinician interface 20. For example, the server 30 may include a web server configured to generate the display screens for presentation upon the clinician display 24. In some embodiments, the clinician display 24 may be configured to present a virtualized desktop that is hosted by the server 30. The server 30 also includes a first communication interface 32 configured to communicate with the clinician interface 20 via a first network 34. In some embodiments, the first network 34 may include a local area network (LAN), such as an Ethernet network. In some embodiments, the first network 34 may include the Internet, and communications between the server 30 and the clinician interface 20 may be secured via encryption, such as, for example, by using a virtual private network (VPN). In some embodiments, the first network 34 may include wired and/or wireless network connections such as Wi-Fi, Bluetooth, ZigBee, Near-Field Communications (NFC), cellular data network, etc. The server 30 includes a first processor 36 and a first machine-readable storage memory 38, which may be called a “memory” for short, holding first instructions 40 for performing the various actions of the server 10 for execution by the first processor 36. The server 10 is configured to store data regarding the treatment plan. For example, the memory 38 includes a system data store 42 configured to hold system data, such as data pertaining to treatment plans for treating one or more patients. The server 10 is also configured to store data regarding performance by a patient in following a treatment plan. For example, the memory 38 includes a patient data store 44 configured to hold patient data, such as data pertaining to the one or more patients, including data representing each patient's performance within the treatment plan.

The system 10 also includes a patient interface 50 configured to communicate information to a patient and to receive feedback from the patient. Specifically, the patient interface includes an input device 52 and an output device 54, which may be collectively called a patient user interface 52, 54. The input device 52 may include one or more devices, such as a keyboard, a mouse, a touch screen input, a gesture sensor, and/or a microphone and processor configured for voice recognition. The output device 54 may take one or more different forms including, for example, a computer monitor or display screen on a tablet, smartphone, or a smart watch. The output device 54 may include other hardware and/or software components such as a projector, virtual reality capability, augmented reality capability, etc. The output device 54 may incorporate various different visual, audio, or other presentation technologies. For example, the output device 54 may include a non-visual display, such as an audio signal, which may include spoken language and/or other sounds such as tones, chimes, and/or melodies, which may signal different conditions and/or directions. The output device 54 may comprise one or more different display screens presenting various data and/or interfaces or controls for use by the patient. The output device 54 may include graphics, which may be presented by a web-based interface and/or by a computer program or application (App.).

As shown in FIG. 1, the patient interface 50 includes a second communication interface 56, which may also be called a remote communication interface configured to communicate with the server 30 and/or the clinician interface 20 via a second network 58. In some embodiments, the second network 58 may include a local area network (LAN), such as an Ethernet network. In some embodiments, the second network 58 may include the Internet, and communications between the patient interface 50 and the server 30 and/or the clinician interface 20 may be secured via encryption, such as, for example, by using a virtual private network (VPN). In some embodiments, the second network 58 may include wired and/or wireless network connections such as Wi-Fi, Bluetooth, ZigBee, Near-Field Communications (NFC), cellular data network, etc. In some embodiments, the second network 58 may be the same as and/or operationally coupled to the first network 34.

The patient interface 50 includes a second processor 60 and a second machine-readable storage memory 62 holding second instructions 64 for execution by the second processor 60 for performing various actions of patient interface 50. The second machine-readable storage memory 62 also includes a local data store 66 configured to hold data, such as data pertaining to a treatment plan and/or patient data, such as data representing a patient's performance within a treatment plan. The patient interface 50 also includes a local communication interface 68 configured to communicate with various devices for use by the patient in the vicinity of the patient interface 50. The local communication interface 68 may include wired and/or wireless communications. In some embodiments, the local communication interface 68 may include a local wireless network such as Wi-Fi, Bluetooth, ZigBee, Near-Field Communications (NFC), cellular data network, etc.

The system 10 also includes a treatment apparatus 70 configured to be manipulated by the patient and/or to manipulate a body part of the patient for performing activities according to the treatment plan. In some embodiments, the treatment apparatus 70 may take the form of an exercise and rehabilitation apparatus configured to perform and/or to aid in the performance of a rehabilitation regimen, which may be an orthopedic rehabilitation regimen, and the treatment includes rehabilitation of a body part of the patient, such as a joint or a bone or a muscle group. The body part may include, for example, a spine, a hand, a foot, a knee, or a shoulder. The body part may include a part of a joint, a bone, or a muscle group, such as one or more vertebrae or a ligament. As shown in FIG. 1, the treatment apparatus 70 includes a controller 72, which may include one or more processors, computer memory, and/or other components. The treatment apparatus 70 also includes a fourth communication interface 74 configured to communicate with the patient interface 50 via the local communication interface 68. The treatment apparatus 70 also includes one or more internal sensors 76 and an actuator 78, such as a motor. The actuator 78 may be used, for example, for moving the patient's body part and/or for resisting forces by the patient.

The internal sensors 76 may measure one or more operating characteristics of the treatment apparatus 70 such as, for example, a force a position, a speed, and/or a velocity. In some embodiments, the internal sensors 76 may include a position sensor configured to measure at least one of a linear motion or an angular motion of a body part of the patient. For example, an internal sensor 76 in the form of a position sensor may measure a distance that the patient is able to move a part of the treatment apparatus 70, where such distance may correspond to a range of motion that the patient's body part is able to achieve. In some embodiments, the internal sensors 76 may include a force sensor configured to measure a force applied by the patient. For example, an internal sensor 76 in the form of a force sensor may measure a force or weight the patient is able to apply, using a particular body part, to the treatment apparatus 70.

The system 10 shown in FIG. 1 also includes an ambulation sensor 82, which communicates with the server 30 via the local communication interface 68 of the patient interface 50. The ambulation sensor 82 may track and store a number of steps taken by the patient. In some embodiments, the ambulation sensor 82 may take the form of a wristband, wristwatch, or smart watch. In some embodiments, the ambulation sensor 82 may be integrated within a phone, such as a smartphone.

The system 10 shown in FIG. 1 also includes a goniometer 84, which communicates with the server 30 via the local communication interface 68 of the patient interface 50. The goniometer 84 measures an angle of the patient's body part. For example, the goniometer 84 may measure the angle of flex of a patient's knee or elbow or shoulder.

The system 10 shown in FIG. 1 also includes a pressure sensor 86, which communicates with the server 30 via the local communication interface 68 of the patient interface 50. The pressure sensor 86 measures an amount of pressure or weight applied by a body part of the patient. For example, pressure sensor 86 may measure an amount of force applied by a patient's foot when pedaling a stationary bike.

The system 10 shown in FIG. 1 also includes a supervisory interface 90 which may be similar or identical to the clinician interface 20. In some embodiments, the supervisory interface 90 may have enhanced functionality beyond what is provided on the clinician interface 20. The supervisory interface 90 may be configured for use by a person having responsibility for the treatment plan, such as an orthopedic surgeon.

The system 10 shown in FIG. 1 also includes a reporting interface 92 which may be similar or identical to the clinician interface 20. In some embodiments, the reporting interface 90 may have less functionality from what is provided on the clinician interface 20. For example, the reporting interface 92 may not have the ability to modify a treatment plan. Such a reporting interface 92 may be used, for example, by a biller to determine the use of the system 10 for billing purposes. In another example, the reporting interface 92 may not have the ability to display patient identifiable information, presenting only pseudonymized data and/or anonymized data for certain data fields concerning a data subject and/or for certain data fields concerning a quasi-identifier of the data subject. Such a reporting interface 92 may be used, for example, by a researcher to determine various effects of a treatment plan on different patients.

In some embodiments, the patient interface 50 and the treatment apparatus 70 are each configured to operate from a patient location geographically separate from a location of the clinician interface 20. For example, the patient interface 50 and the treatment apparatus 70 may be used as part of an in-home rehabilitation system, which may be monitored remotely by using the clinician interface 20 at a centralized location, such as a clinic or hospital. In some embodiments, either or both of the patient interface 50 and/or the treatment apparatus 70 are configured to communicate with a remote computer, such as the server 30, to receive the treatment plan and to report back to the remote computer with data regarding performance by the patient in following the treatment plan.

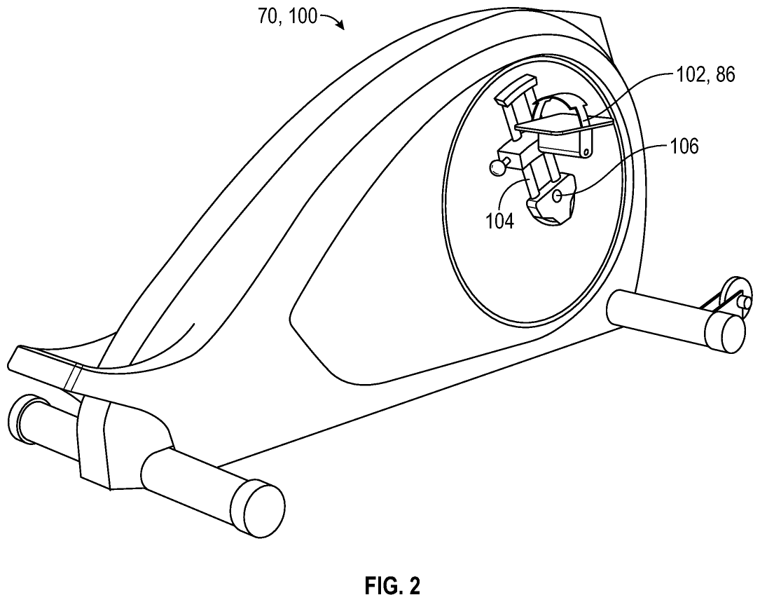

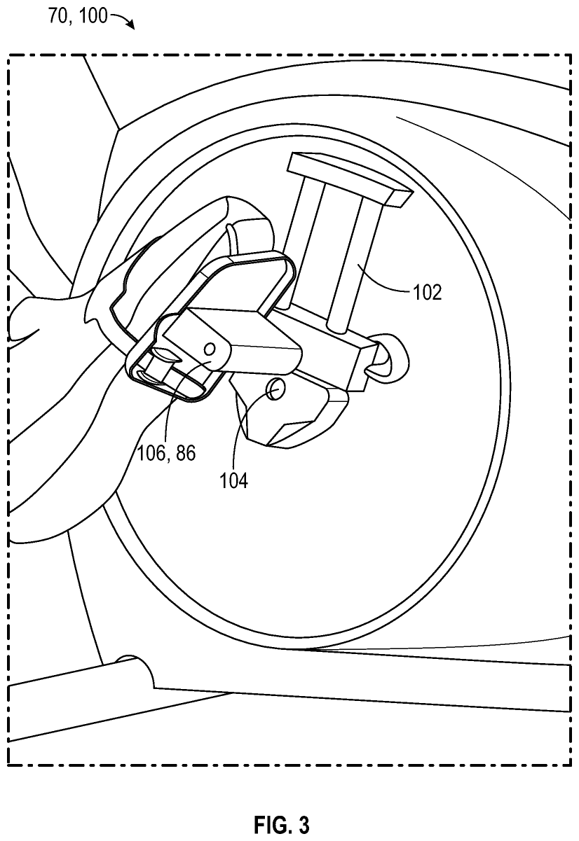

FIGS. 2-3 show an embodiment of a treatment apparatus 70. More specifically, FIG. 2 shows a treatment apparatus 70 in the form of a stationary cycling machine 100, which may be called a stationary bike, for short. The stationary cycling machine 100 includes a set of pedals 102 each attached to a pedal arm 104 for rotation about an axle 106. In some embodiments, and as shown in FIG. 2, the pedals 102 are movable on the pedal arms 104 in order to adjust a range of motion used by the patient in pedaling. For example, the pedals being located inwardly toward the axle 106 corresponds to a smaller range of motion than when the pedals are located outwardly away from the axle 106. A pressure sensor 86 is attached to or embedded within one of the pedals 106 for measuring an amount of force applied by the patient on the pedal 106. The pressure sensor 86 may communicate wirelessly to the treatment apparatus 70 and/or to the patient interface 50.

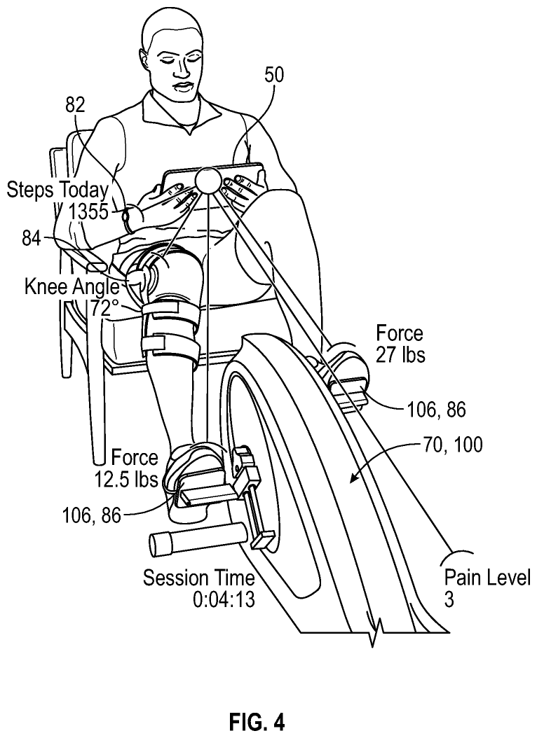

FIG. 4 shows a person (a patient) using the treatment apparatus of FIG. 2, and showing sensors and various data parameters connected to a patient interface 50. The example patient interface 50 is a tablet computer or smartphone, or a phablet, such as an iPad, an iPhone, an Android device, or a Surface tablet, which is held manually by the patient. In some other embodiments, the patient interface 50 may be embedded within or attached to the treatment apparatus 70. FIG. 4 shows the patient wearing the ambulation sensor 82 on his wrist, with a note showing “STEPS TODAY 1355”, indicating that the ambulation sensor 82 has recorded and transmitted that step count to the patient interface 50. FIG. 4 also shows the patient wearing the goniometer 84 on his right knee, with a note showing “KNEE ANGLE 72°”, indicating that the goniometer 84 is measuring and transmitting that knee angle to the patient interface 50. FIG. 4 also shows a right side of one of the pedals 106 with a pressure sensor 86 showing “FORCE 12.5 lbs.,” indicating that the right pedal pressure sensor 86 is measuring and transmitting that force measurement to the patient interface 50. FIG. 4 also shows a left side of one of the pedals 106 with a pressure sensor 86 showing “FORCE 27 lbs.”, indicating that the left pedal pressure sensor 86 is measuring and transmitting that force measurement to the patient interface 50. FIG. 4 also shows other patient data, such as an indicator of “SESSION TIME 0:04:13”, indicating that the patient has been using the treatment apparatus 70 for 4 minutes and 13 seconds. This session time may be determined by the patient interface 50 based on information received from the treatment apparatus 70. FIG. 4 also shows an indicator showing “PAIN LEVEL 3”. Such a pain level may be obtained from the patent in response to a solicitation, such as a question, presented upon the patient interface 50.

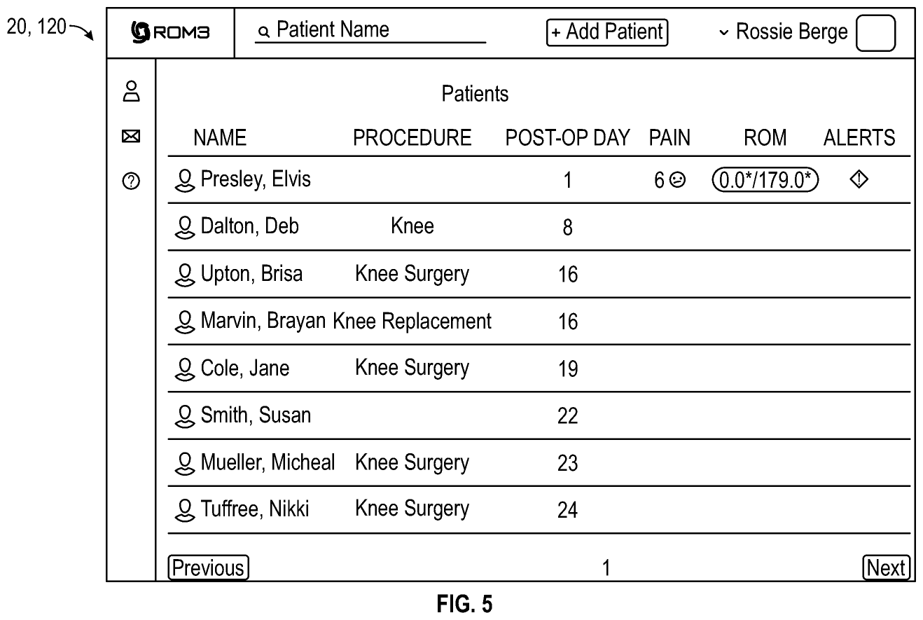

FIG. 5 is an example embodiment of an overview display 120 of the clinician interface 20. Specifically, the overview display 120 presents summary information regarding each of a plurality of different patients. In some embodiments, and as shown on FIG. 5, the summary information includes an indicator showing a procedure performed upon each of the patients, temporal progress of the patient within the treatment plan (post-op day), an indicator of a last-reported pain level, range-of-motion (ROM) numbers, and an indicator showing if there are any alerts requiring special attention.

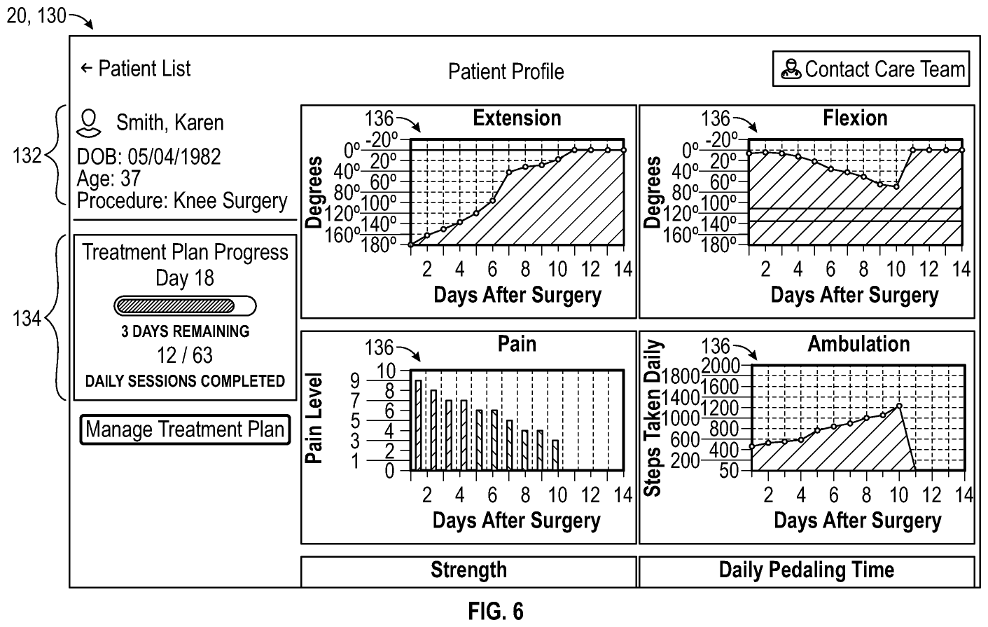

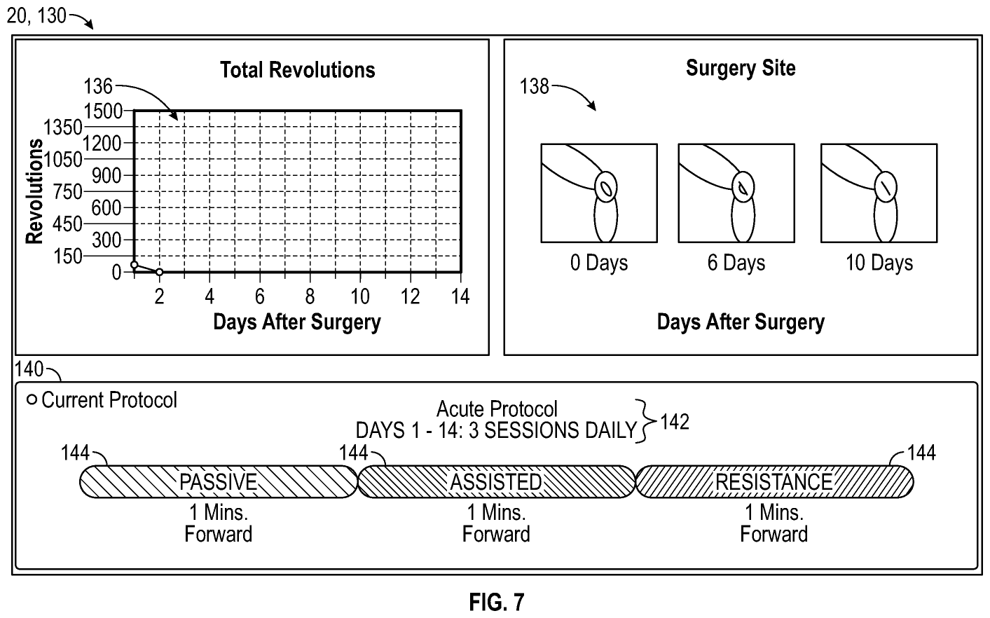

FIGS. 6-7 show an example embodiment of a patient profile display 130 of the clinician interface 20. The example patient profile display 130 includes a patient summary 132 with the patient's name, date of birth (DOB), age, a description of a procedure performed or to be performed on the patient, e.g., “Knee surgery”, and a picture of the patient, if available. The example patient profile display 130 also includes a treatment progress summary 134, showing one or more indicators of progress within a treatment regimen or plan. The example treatment progress summary 134 shown on FIG. 6 includes textual progress summaries, “DAY 18”, “3 days remaining”, “12/63 DAILY SESSIONS COMPLETED”, as well as graphical progress summaries in the form of horizontal bar graphs, which may also be called progress bars.

The example patient profile display 130 presents information regarding a treatment history of the patient. For example, the example patient profile display 130 includes a plurality of different treatment graphs 136 showing the effect of various treatment parameters over time. The treatment graphs 136 shown in the example patient profile display 130 of FIGS. 6-7 include extension (angle), flexion (angle), pain (0-10 scale), ambulation (steps/day), and total revolutions (i.e., revolutions performed on the stationary cycling machine 100). The patient profile display 130 shown on FIG. 7 also includes a pictorial history 138, showing one or more images of the surgical site for reference by a clinician or other healthcare professional in reviewing post-operative progress. The images in the pictorial history 138 may be taken by the patient and/or by a clinician or other healthcare professional. For example, the first picture may be taken by a member of the surgical staff, and subsequent pictures may be taken by the patient and/or the rehabilitation clinician. The example patient profile display 130 shown on FIG. 7 also includes a protocol summary display 140 showing a summary overview of a treatment protocol to be performed by the patient. The example protocol summary display 140 includes a protocol heading 142 with a protocol name, e.g. “Acute Protocol.” The protocol heading 142 also includes overview information regarding how and when the protocol is to be performed, e.g. “Days 1-14, 3 sessions daily.” The protocol summary display 140 also includes several protocol session icons 144, each indicating details of an activity to be performed within a protocol session, e.g., “Passive”, “Active”, or “Resistance”, together with other information regarding the protocol session, such as a direction (forward/reverse), and an amount of time that each protocol session is prescribed to be performed.

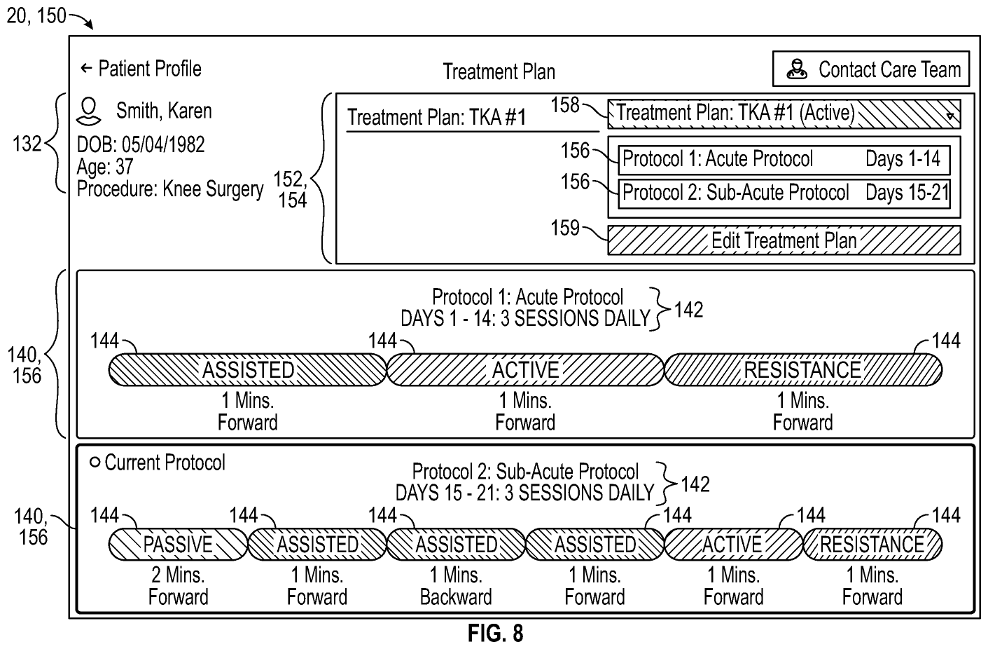

FIG. 8 shows an example embodiment of a treatment plan management display 150 of a clinician interface 20 presenting a treatment protocol 156 for the patient. More specifically, FIG. 8 shows a treatment plan display 150, which includes a treatment plan overview 152 for displaying and modifying a treatment plan 154, which itself comprises one or more different treatment protocols 156. In some embodiments, the treatment plan 154 may be a rehabilitation regimen for rehabilitation treatment of a particular body part, such as a joint or a bone or a muscle group. In some embodiments, the treatment plan 154 may be a weight loss or muscle-gaining regimen for working various different body parts to improve overall health and/or fitness.

The treatment plan overview 152 includes a plan selection control 158 in the form of a drop-down control for a clinician to select a given treatment plan 154. However, other different controls and/or presentations may be provided for viewing and/or modifying a treatment plan 154. The example treatment plan 154 shown on FIG. 8 includes a plan name (TKA #1), and a listing of the treatment protocols 156 that comprise the treatment plan 154. The treatment plan overview 152 shown on FIG. 8 also includes an edit plan control 159 in the form of a button for invoking a plan modification control 160 for allowing the clinician to modify the treatment plan 154.

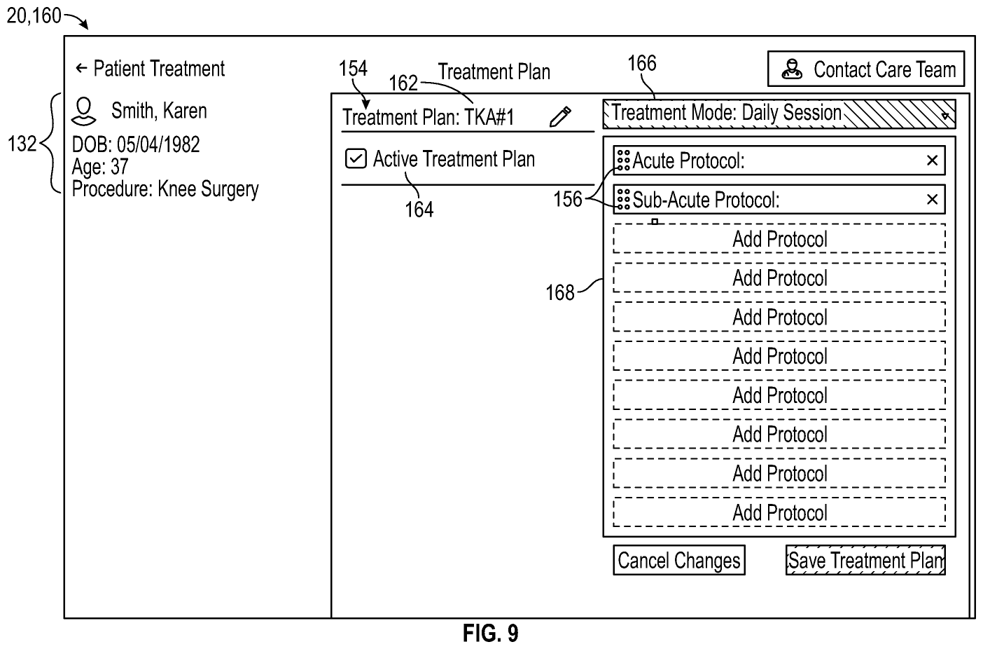

FIG. 9 shows an example embodiment of the plan modification control 160 of the clinician interface 20. The example plan modification control 160 shown on FIG. 9 is a full-screen display, but it could take other forms such as, for example, a popup window or a region or section of another display screen. The plan modification control 160 includes a control 162 for renaming the treatment plan 154. The plan modification control 160 also includes an active plan control 164 for setting the treatment plan 154 as an active treatment plan for active use and for setting the treatment plan 154 as not an active treatment plan. The plan modification control 160 also includes a treatment mode selector 166 for changing a treatment mode of the treatment plan 154. The treatment modes may include different settings for the frequency of sessions to be performed. For example, the treatment modes may include “Daily Session” or “3× per day sessions.” The plan modification control 160 is also configured to modify the treatment protocols 156 within the treatment plan 154. Specifically, the plan modification control 160 includes a plan configuration control 168 for changing the treatment protocols 156 within the treatment plan 154. Thus, the plan modification control 160 is configured to modify the plurality of treatment protocols 156 that comprise the treatment plan 154.

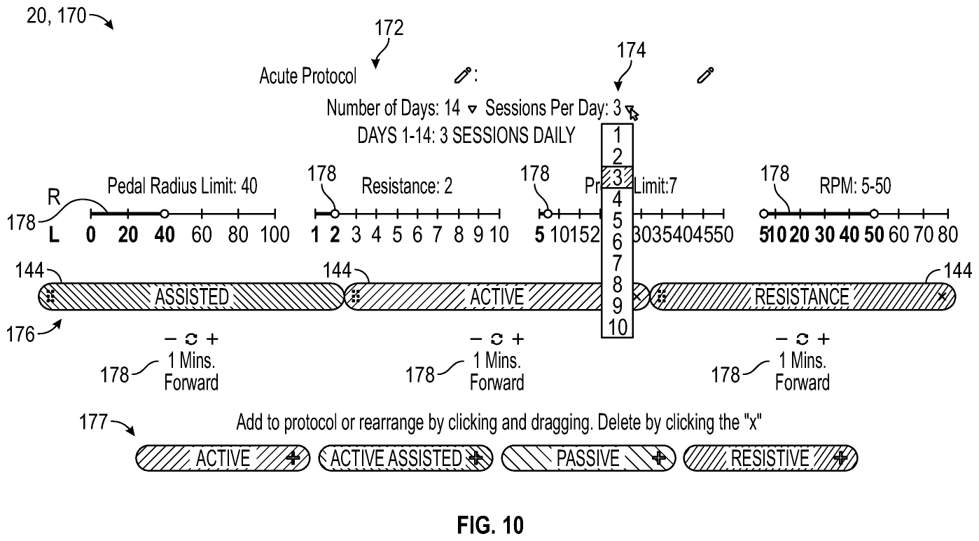

FIG. 10 shows an example embodiment of a protocol management display 170 of a clinician interface 20 for editing a treatment protocol 156. Specifically, the protocol management display 170 includes a protocol name control 172 for renaming the treatment protocol 156. The protocol management display 170 also includes a protocol timing control 174 for adjusting various timing settings of the treatment protocol 156, such as a duration for the treatment protocol 156 within the treatment plan 152, and a number of sessions to be performed per day. The example protocol timing control 174 shown on FIG. 10 includes drop-down menus for changing the various timing settings, but other controls could be used such as, for example, numeric entry fields or increase/decrease buttons. The protocol management display 170 also includes a protocol session control 176 for customizing the session periods. Specifically, the protocol session control 176 includes a graphical representation of a session, with protocol session icons 144, which may be similar or identical to the protocol session icons 144 of the protocol summary display 140. Each session period may have an associated type, such as passive, resistance, assisted, or active. Each session period may also have several parameters associated therewith.

The protocol session control 176 allows the clinician to adjust the number, the order, and the types of the session periods within a given session of the treatment protocol 156. Each session period has a type that corresponds to a category of activity to be performed upon a body part during that session period. For example, the session periods may be one of a passive period, an assisted period, an active period, or a resistance period. Each passive period is associated with a particular activity that includes moving a body part by an external force; each assisted period is associated with a particular activity that includes moving the body part by the patient with assistance of the external force; each active period is associated with a particular activity that includes the patient moving the body part without assistance of the external force; and each resistance period is associated with a particular activity that includes the patient actively moving the body part against a resistance force. For example, where the treatment apparatus 70 includes a stationary cycling machine 100, a passive period may include an actuator 78, such as a motor, that rotates the pedals 108 with the patient's feet and legs attached thereto and without any action or force being applied by the patient. An assisted period may include the patient applying force to rotate the pedals 108 with some additional help or assistance from the actuator 78. An active period may include the patient applying force to rotate the pedals 108 without any assistance from any outside force. A resistance period may include the patient exerting some force to rotate the pedals 108 in opposition to a resistance force applied by the actuator 78. In some embodiments, the actuator 78 may produce the external forces for each of the different categories of the session periods. The external forces may have different attributes, such as directions, intensities, or rates of changes, for each of the different categories of the session periods. Each session may include any number of session periods in any combination.

In some embodiments, the protocol session icons 144 may be modified using a drag-and-drop interface. Additional protocol sessions may be added to the protocol session using a session period control 177. Additionally, parameters for any or all of the session periods may be adjusted using various session parameter controls 178. For example, a duration and direction of each session period may be adjusted using the session parameter controls 178 located below an associated one of the protocol session icons 144. Various other parameters, such as resistance, target speed range (RPM), pedal radius limits, etc. may be adjusted using other session parameter controls 178. In some embodiments, the number and the type of session parameter controls 178 may change depending on the type of session period selected. For example, selecting a protocol session icon 144 for an active type of session period may cause the target speed range (RPM) session parameter control 178 to be visible and adjustable, but the target speed range (RPM) session parameter control 178 may not be visible and/or adjustable in response to selecting a protocol session icon 144 for a passive type session.

In some embodiments, the system 10 may impose limits on values that can be set using the session parameter controls 178. For example, the treatment plan 154 may include a maximum session time. In some embodiments, to satisfy a rule of the system 10 or a rule within the treatment plan 154, one or more of the values of the parameters may be automatically changed by the system 10. For example, the treatment plan 154 may require a resistance type of session period after an active type of session period, wherein the former is at least 25% as long as the active type of session to allow the patient to cool down after active exercise. The system 10 may automatically create the resistance type session period in response to the clinician creating an active type session period. The system 10 may also automatically adjust the time of the resistance type session period to satisfy the requirement of it lasting at least 25% as long as the active type of session.

In some embodiments, the treatment plan 154 may include maximum values for certain parameters until an associated condition is satisfied. For example, the pedal radius limit may be limited to 40 mm until an associated condition is satisfied. Associated conditions may include, for example, approval by an authorized person, such as an orthopedic surgeon; the elapsing of a particular time, such as 5 days after a surgical procedure; or successful completion of a post-operation checkup. Similarly, the treatment plan 154 may place limits on the types of session periods that may be performed until an associated condition is satisfied. The treatment plan 154 may be limited to only passive or assisted session periods (and not active periods or resistance periods until an associated condition is satisfied. Different associated conditions may be associated with each of the different parameters and/or with limits on the types of session periods available.

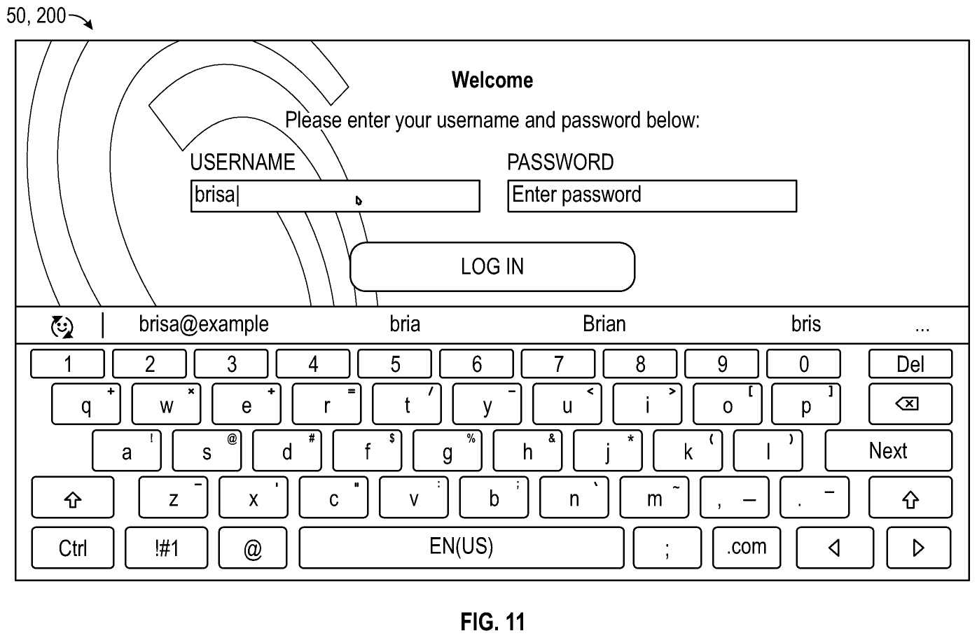

FIG. 11 shows an example embodiment of a login screen 200 of the patient interface 50. The login screen 200 may allow the patient to identify himself or herself to the system 10 and to verify their identity prior to using the system for treatment. In some embodiments, the patient interface 50 may not have a login screen 200. For example, the patient interface 50 may be preconfigured for a given patient. This may be used, for example, where the patient interface 50 is provided for in-home care by a single patient. Additionally or alternatively, another means may be used for the patient to identify himself or herself to the system 10. For example, a radio-frequency identification (RFID) device, such as a keycard or a wristband, may be placed upon or near a reader coupled to the patient interface 50. In another example, the patient interface 50 may use a biometric identification, such as a fingerprint, a palm print, or a retinal scan, to verify the identity of a patient.

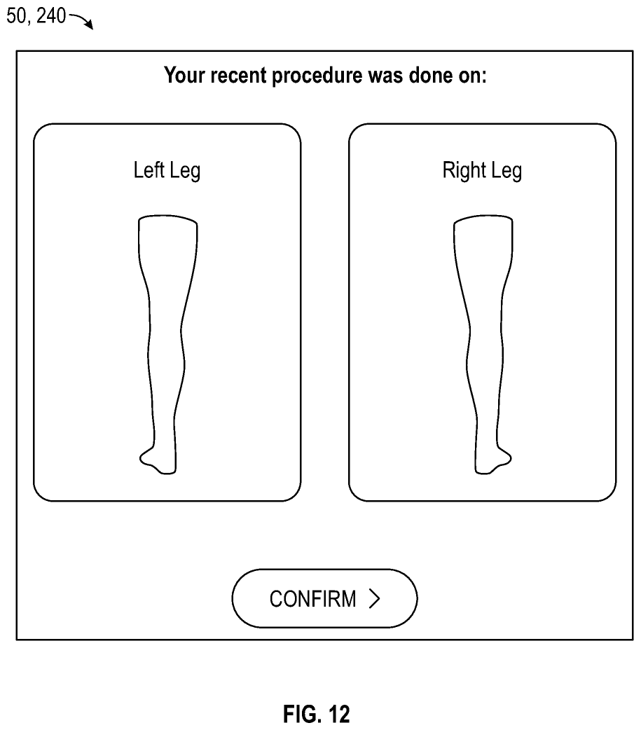

FIG. 12 shows an example of a body part confirmation screen 240 of the patient interface 50. The patient may be asked to confirm the particular body part to be treated in order to ensure that the treatment plan is correct for that particular patient. The body part confirmation screen 240 is also used to ensure that the patient is aware of the body part to be treated (i.e., to set the patient's expectations) to minimize risk of an unexpected event that could result in an adverse outcome. If the patient selects a body part that does not comport with the treatment plan, the treatment session may be cancelled, and a notice to the clinician may be generated. The patient may be asked to confirm their selection and, then, given the opportunity to change their selection to ensure that treatment is not cancelled and/or that the clinician is not notified in case of a mis-selection.

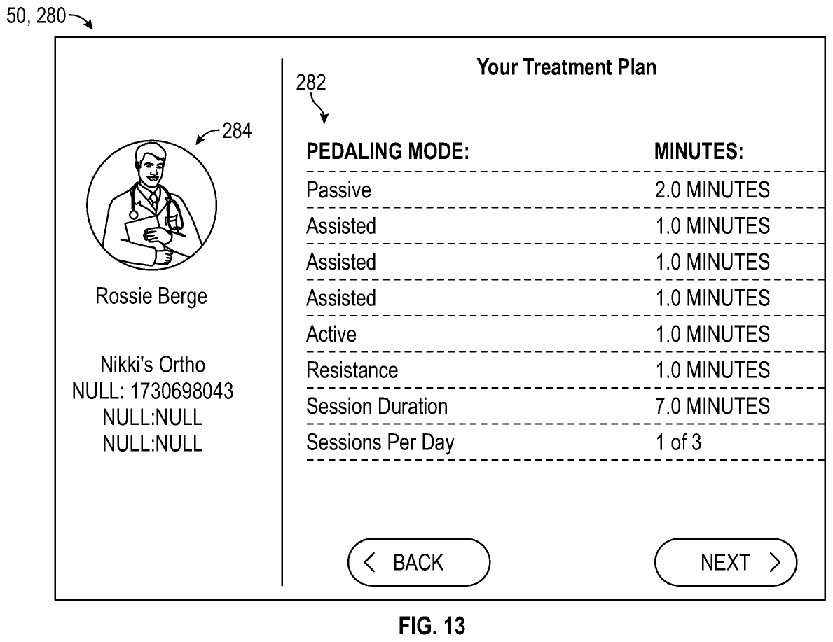

FIG. 13 shows an example of a treatment plan overview screen 280 of the patient interface 50. Specifically, the treatment plan overview screen 280 includes a session summary 282 with a listing of the session periods and their respective durations (in minutes), with the total session duration, and with a display of the sessions per day. The treatment plan overview screen 280 includes a practitioner contact data 284 with a picture and phone numbers for a practitioner that the patient may contact for help. The practitioner may be, for example, an orthopedic surgeon and/or a physical therapist.

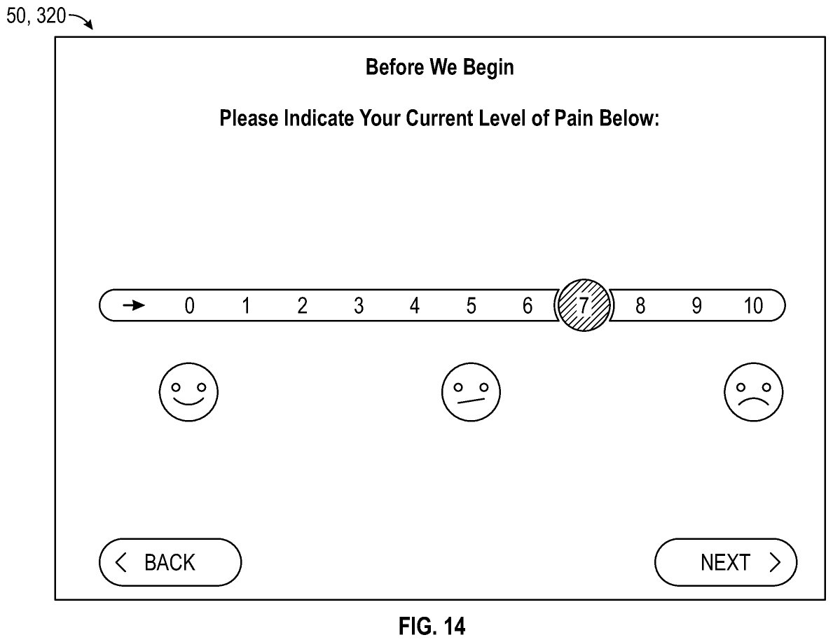

FIG. 14 shows an example embodiment of a pain level input screen 320 of the patient interface 50. The pain level input screen 320 includes a pain level selector 322 for the patient to indicate his or her pain level. The example pain level selector 322 is a slider that the patient can use to select a pain level of 0 (no pain) to 10 (maximum pain). The pain level input screen 320 may include other inputs, such as a numeric input or check boxes. Graphics, such as happy-to-sad face icons may help to clarify that 10 is the highest level of pain. A similar pain level input screen 320 may be presented before, during, and/or after a treatment session for tracking the effect of the session on the patient's pain level.

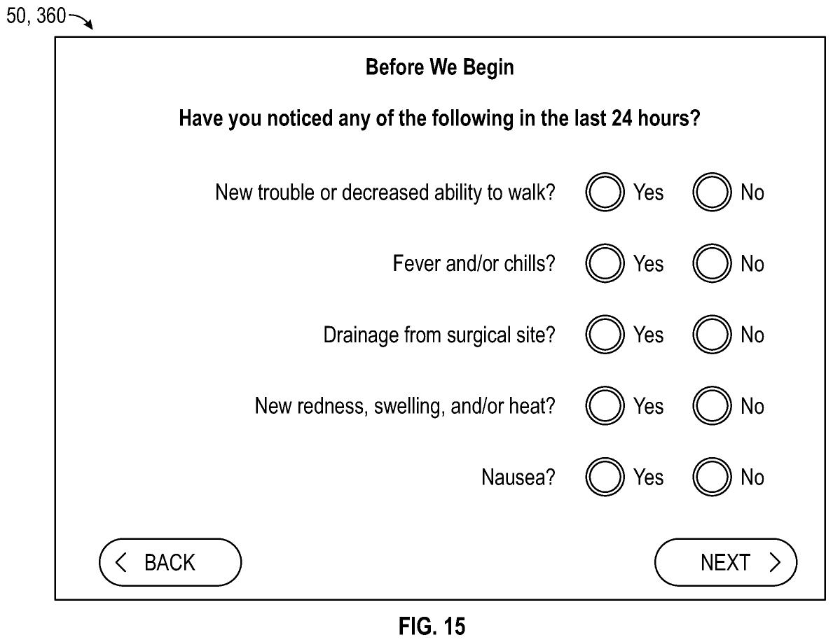

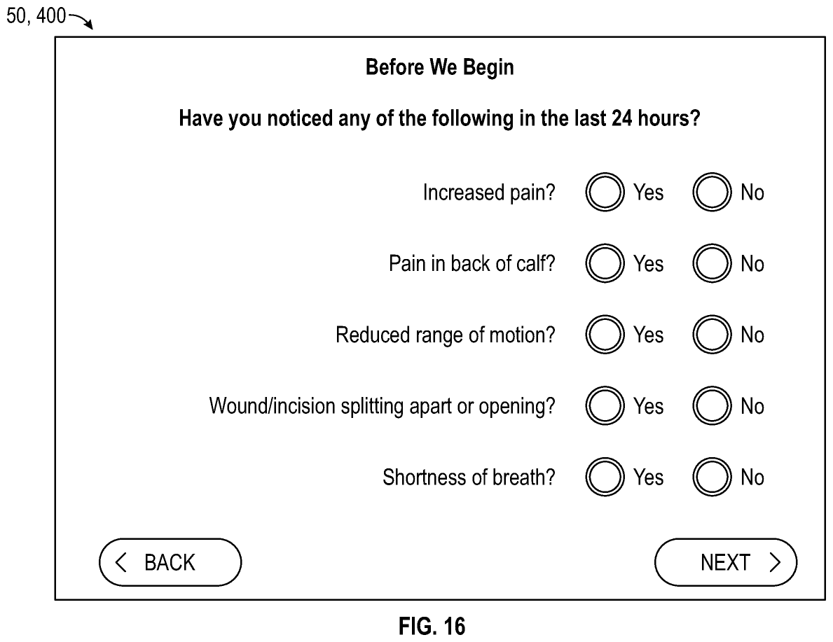

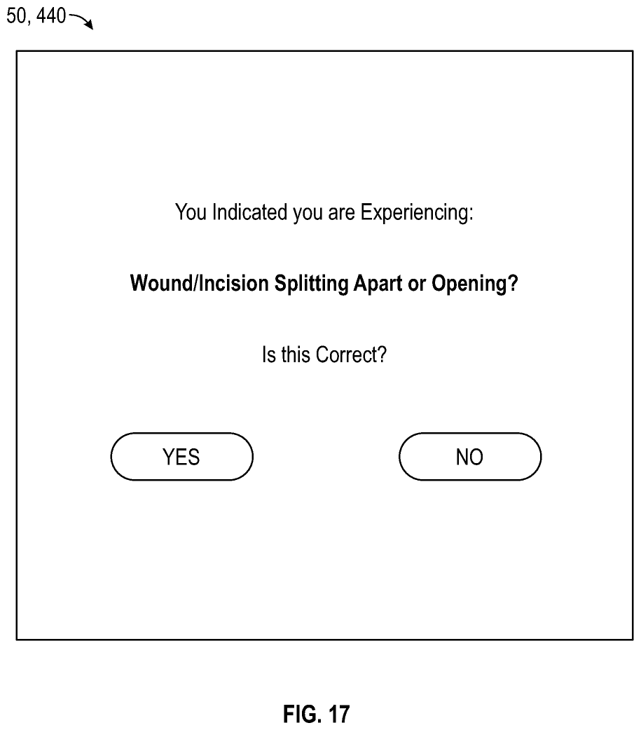

FIGS. 15-16 show an example embodiment of a first patient questionnaire screen 360 and a second patient questionnaire screen 400 of the patient interface 50, respectively. The patient questionnaire screens 360, 400 may help to determine if the patient has any conditions that may adversely impact their ability to perform the training session. In some embodiments, particular answers to one or more of the questions may trigger associated actions, such as alerting the clinician and/or locking the treatment apparatus to prevent it from being used until the clinician or another authorized person is able to verify that it is safe for the patient to proceed with the training session. For example, affirmative answers to either of the “[Have you noticed] wound/incision splitting” and/or the “[Have you noticed] shortness of breath” inquiries may prevent the patient from proceeding with the treatment until the clinician first verifies that it is safe for the patient to proceed. A question confirmation screen 440, such as the one shown in FIG. 17, may be used before the system locks the treatment apparatus and/or generates an alert to prevent those actions in case the patient indicates an associated condition, such as “shortness of breath,” in error.

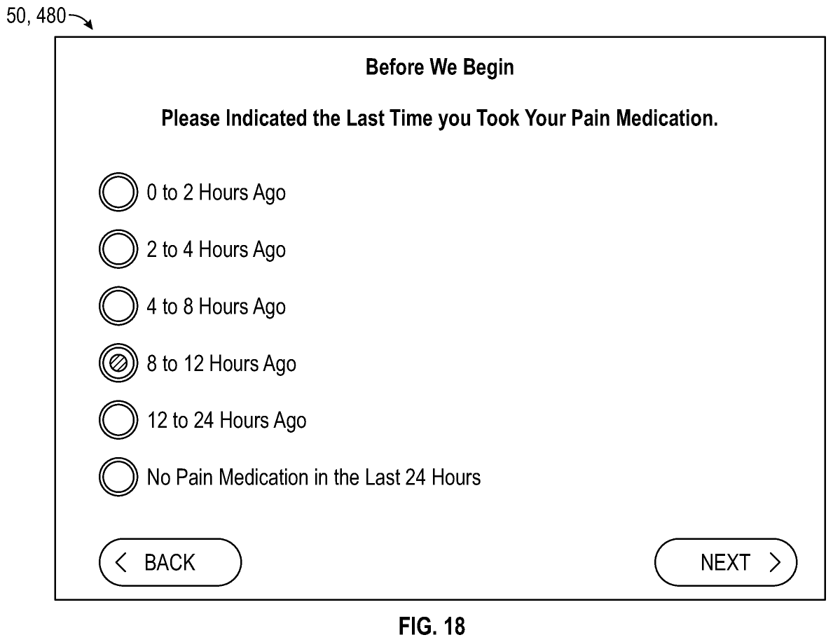

FIG. 18 shows an example embodiment of a third patient questionnaire screen 480 of the patient interface 50. Specifically, the third patient questionnaire screen 480 requests the patient to indicate when they last took pain medication. This question may be used to compensate for the effects of pain medication on reported pain level and/or other performance parameters.

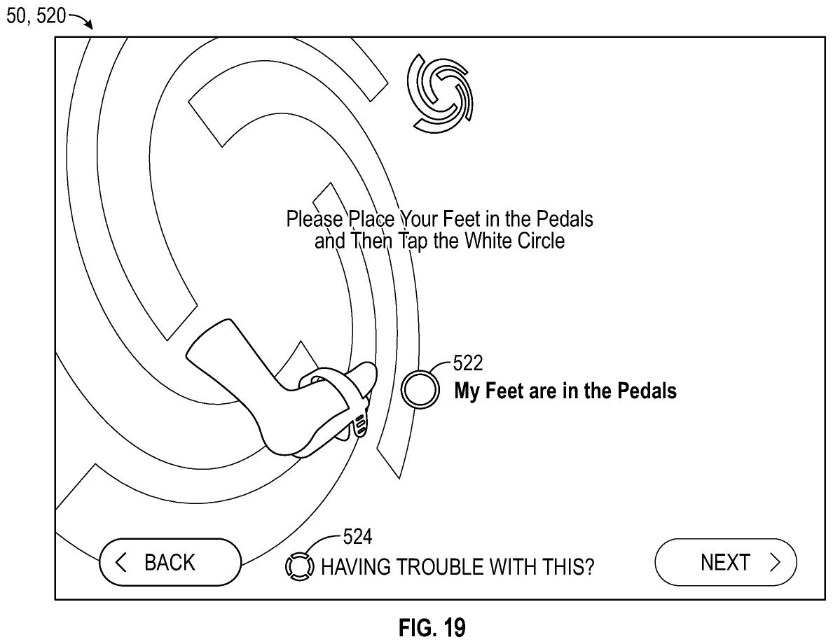

FIG. 19 shows an example embodiment of positioning confirmation screen 520 of the patient interface 50. This screen 520 is the beginning of a guided walk-through for the patient to use the treatment apparatus 70. Specifically, this screen 520 includes written instructions to guide the patient in placing their feet in the pedals 102 of a stationary cycling machine 100. In some embodiments, this screen 520 may include graphics, such as pictures or animations to help the patient perform particular actions for using the treatment apparatus 70. Screen 520 includes a position confirmation selector 522 for the patient to indicate that they are in position to use the treatment apparatus 70. Screen 520 also includes a trouble button 524 for the patient to indicate that they are having trouble getting in position to use the treatment apparatus 70.

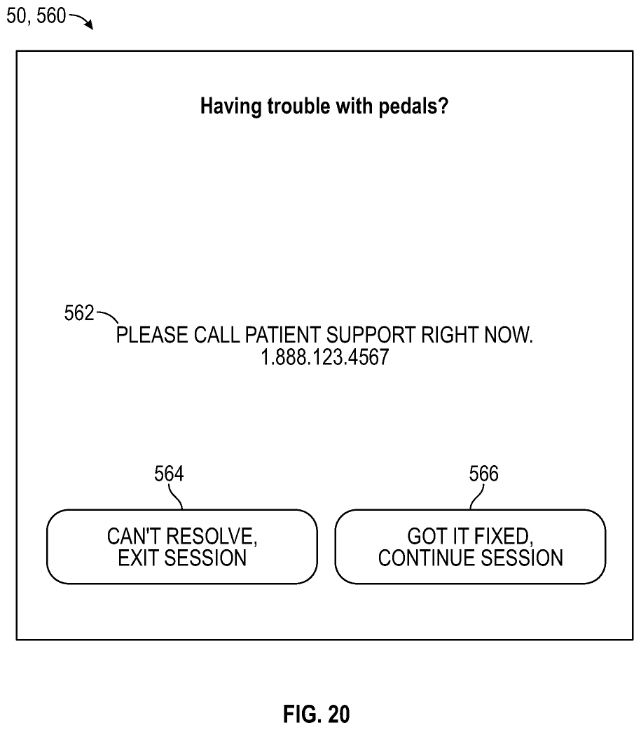

FIG. 20 shows an example embodiment of a positioning help screen 560 of the patient interface 50. This help screen 560 may be shown in response to the user selecting the trouble button 524 on the positioning confirmation screen 520. The help screen 560 may automatically be displayed if the patient fails to select the position confirmation selector 522 within a predetermined period of time. In some embodiments, an intermediate screen such as a popup asking if the patient needs more time may be displayed before the help screen 560 is shown. The help screen 560 includes assistance instructions 562 for the patient to obtain assistance for using the treatment apparatus 70. In some embodiments, the assistance instructions 562 may include a phone number. The assistance instructions 562 may also include other items, such as a link to a video conference with someone able to help the patient, and/or a link to a video or animated walk-through with detailed instructions for performing a particular action to use the treatment apparatus 70. The particular action may include, for example, placing the feet in the pedals. The help screen 560 may also include an exit button 564 that the patient can use to stop the treatment session in case they are unable to resolve their issue with using the treatment apparatus 70. Use of the exit button 564 may generate an alert to the clinician. The help screen 560 also includes a proceed button 566 that the patient can use to indicate that they have resolved their issue and are able to proceed with the treatment session.

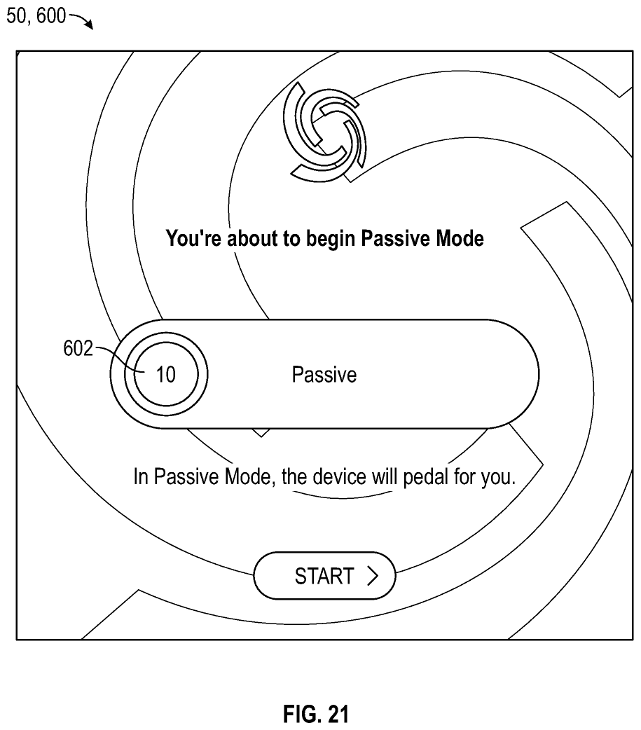

FIG. 21 shows an example embodiment of a session period introduction screen 600 of the patient interface 50. The session period introduction screen 600 includes various description text and/or graphics to inform the patient of the type of the session period about to begin. The session period introduction screen 600 may also include a countdown timer 602 to aid the patient in preparing to begin the forthcoming session period.

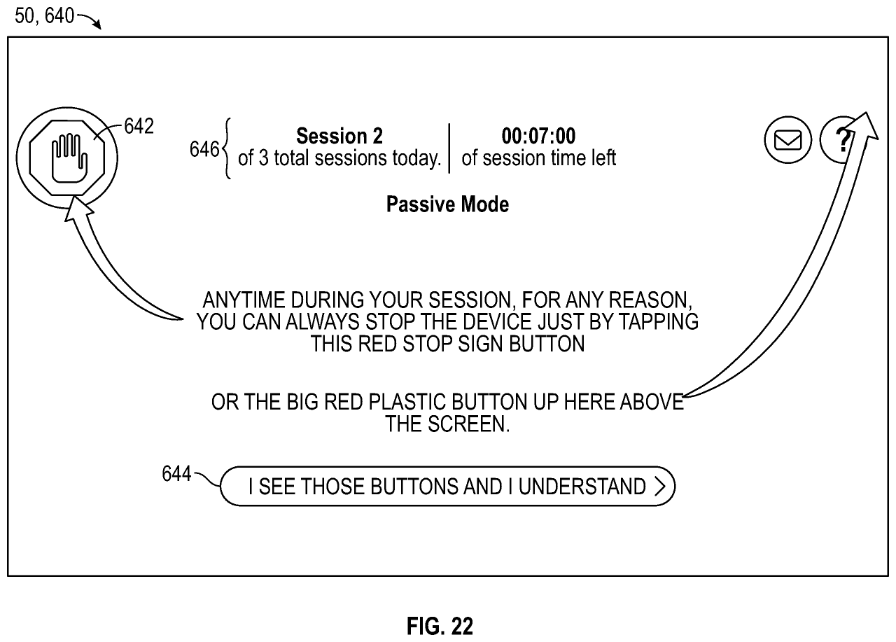

FIG. 22 shows an example embodiment of an emergency stop (E-stop) confirmation screen 640 of the patient interface 50. The E-stop confirmation screen 640 introduces an on-screen E-stop control 642 as a stop button present on all screens of the patient interface 50 during the treatment session for stopping the treatment apparatus 70. The E-stop confirmation screen 640 explains the use of the E-stop control 642 and the use of a physical E-Stop button (not shown) located on or near the patient interface 50. The E-stop confirmation screen 640 also includes a confirmation button 644 that, before the treatment session may begin, the patient must select to confirm that they understand how to use the E-stop control 642 and the physical E-Stop button. The E-stop confirmation screen 640 and other screens of the patient interface 50 also include a session overview display 646 with progress information regarding the current session, such as a session number for the day, the remaining time, and/or the elapsed time of the session.

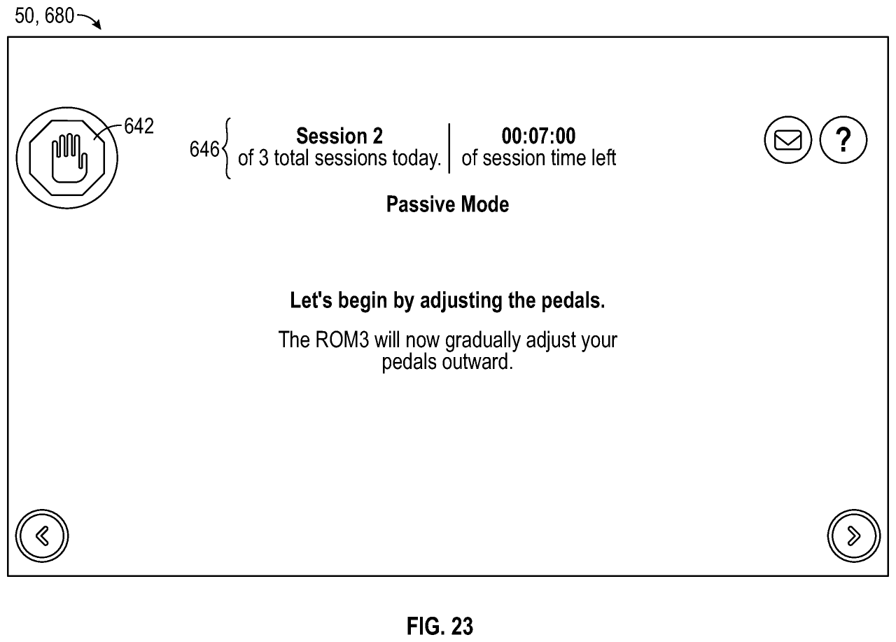

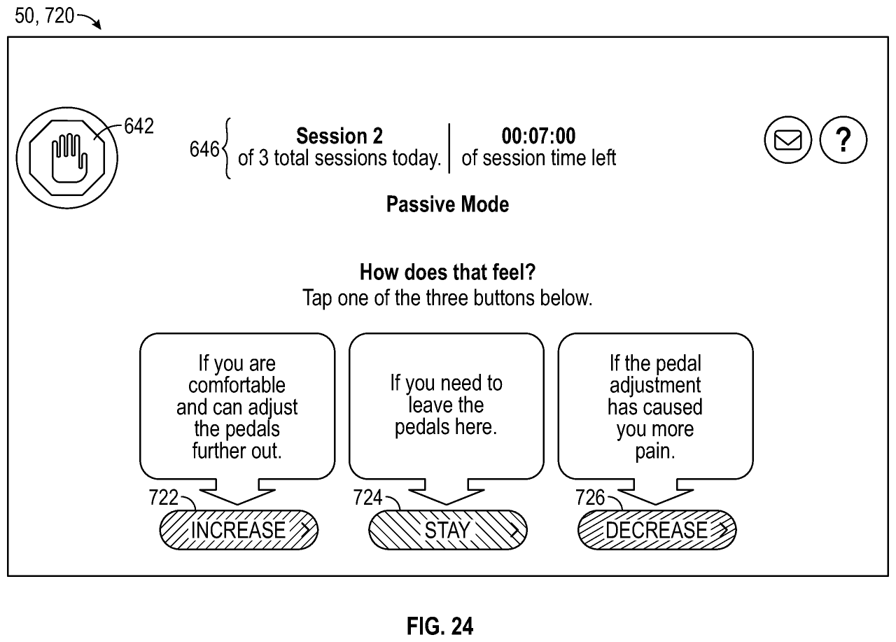

FIG. 23 shows an example embodiment of an adjustment introduction screen 680 of the patient interface 50. The adjustment introduction screen 680 includes text and/or graphics indicating various adjustments to be performed by the treatment apparatus 70. In the example shown, the adjustments include the treatment apparatus 70 that is a stationary cycling machine 100 that automatically moves the pedals 102 outwardly to a predetermined position for the session period. FIG. 24 shows an example embodiment of an adjustment confirmation screen 720 of the patient interface 50. The adjustment confirmation screen 720 includes text and/or graphics requesting the patient to confirm their satisfaction with the position of the treatment apparatus 70 during and/or after the automatic adjustments are made. The adjustment confirmation screen 720 includes an increase button 722 that the patient may select to indicate a desire to change the position of the treatment apparatus 70 such as, for example, to increase the radius of the pedal 102 on the pedal arm 104. The adjustment confirmation screen 720 also includes a stay button 724 that the patient may select to indicate acceptance of the position of the treatment apparatus 70. The adjustment confirmation screen 720 also includes a decrease button 726 that the patient may select to indicate a desire to change the position of the treatment apparatus 70. For example, if the patient experiences pain or discomfort with the initial position, he or she may change the position using the decrease button 726 until the pain or discomfort is alleviated.

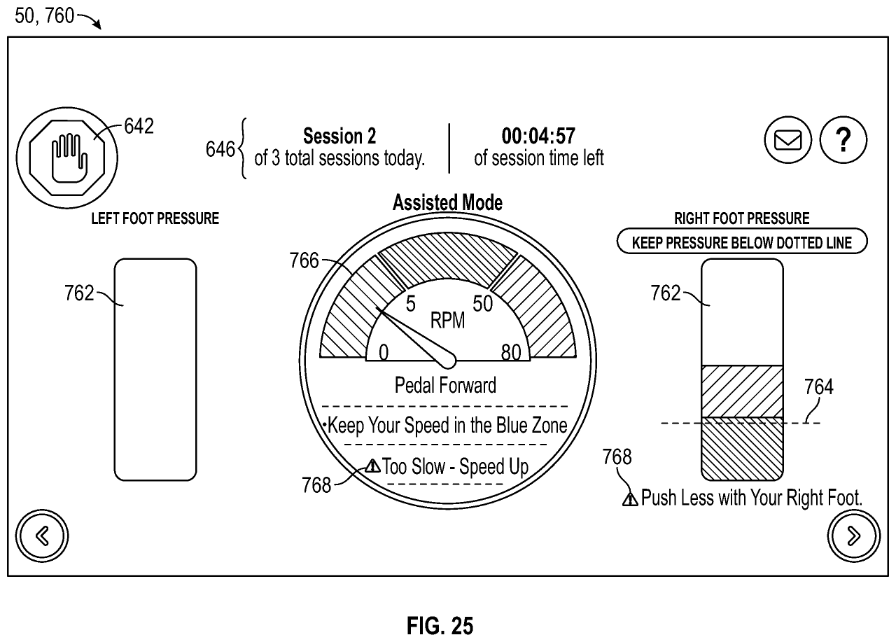

FIG. 25 shows an example embodiment of a session period action screen 760 of the patient interface 50. This screen 760 is displayed while a given session period is in progress. It includes pressure indicators 762 showing an amount of pressure or force applied by each foot. The pressure indicators 762 show the pressures of the patient's feet upon the pedals 106 as measured by the pressure sensors 86. The pressure indicators 762 are shown as bar graphs, but other types of displays may be used, such as rotary gauges and/or numeric indicators. The pressure indicators 762 may also include a target pressure indicator 764 representing a target pressure value which may be determined by the clinician using an associated session parameter control 178 on the protocol management display 170, as shown, for example, on FIG. 10.

The session period action screen 760 also includes a speed indicator 766 showing a speed that the pedals 106 are turning, as measured by an internal sensor 76 of the stationary cycling machine 100. The speed indicator 766 is shown as a rotary gauge, but other types of displays may be used, such as a bar graph and/or a numeric indicator. The speed indicator 766 includes an optimal or desired speed range, which may be determined by the clinician using an associated session parameter control 178 on the protocol management display 170, as shown, for example, on FIG. 10. The session period action screen 760 may present prompts or messages 768 to enable the user to change the pressure and/or speed if either of those parameters is outside of a predetermined range.

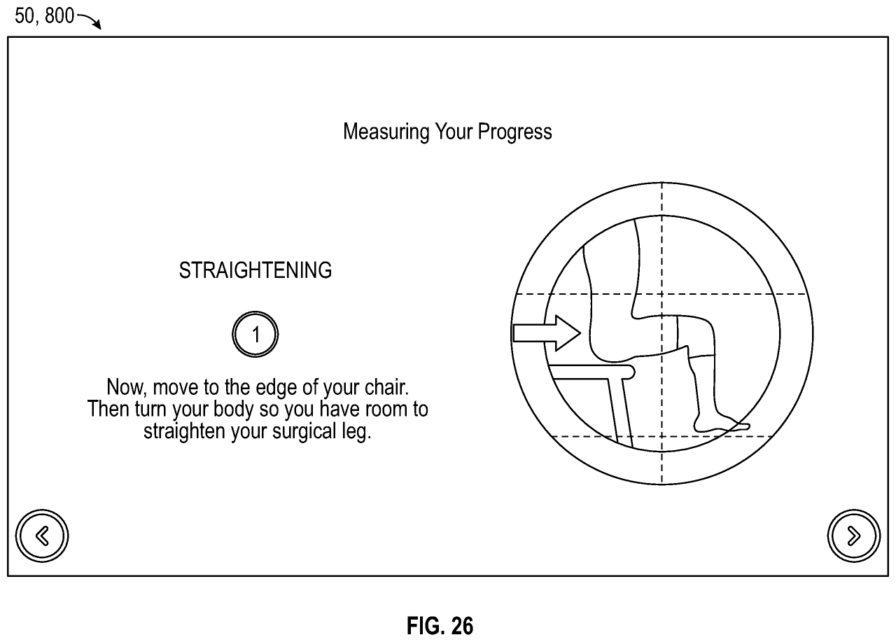

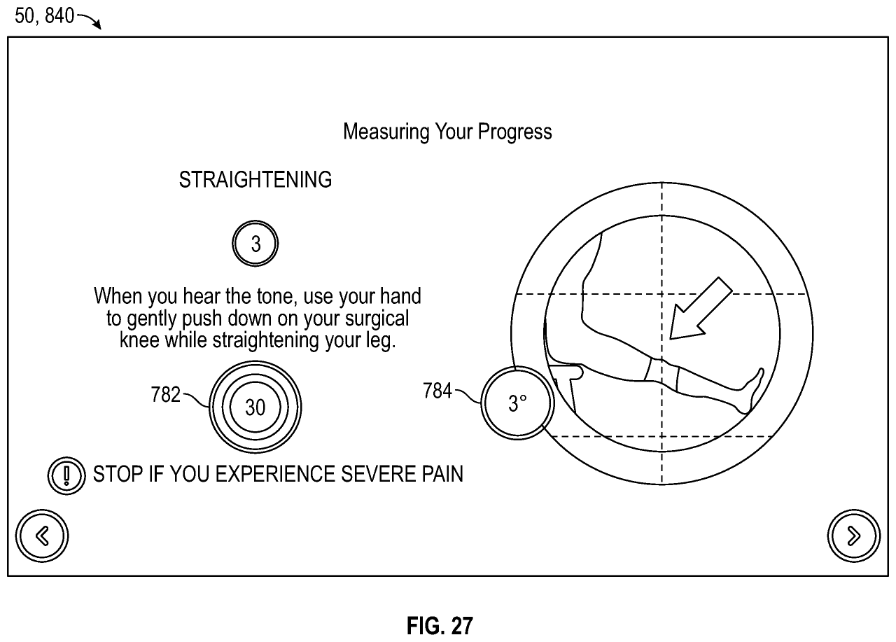

FIG. 26 shows an example embodiment of an exercise introduction screen 800 of the patient interface 50. The exercise introduction screen 800 includes instructions and/or prompts for the patient to perform an exercise that is not performed using the treatment apparatus 70. In the example shown on FIG. 26, the exercise involves straightening the patient's leg. FIG. 27 shows an example embodiment of an exercise action screen 840 of the patient interface 50. The exercise action screen 840 includes a countdown timer 842 showing an amount of time that the patient should continue with a given exercise. The exercise action screen 840 also includes an angle display 844 showing an angle of a body part being exercised. The angle display 844 may show, for example, a knee flex angle measured by the goniometer 84 that is attached to the patient's knee.

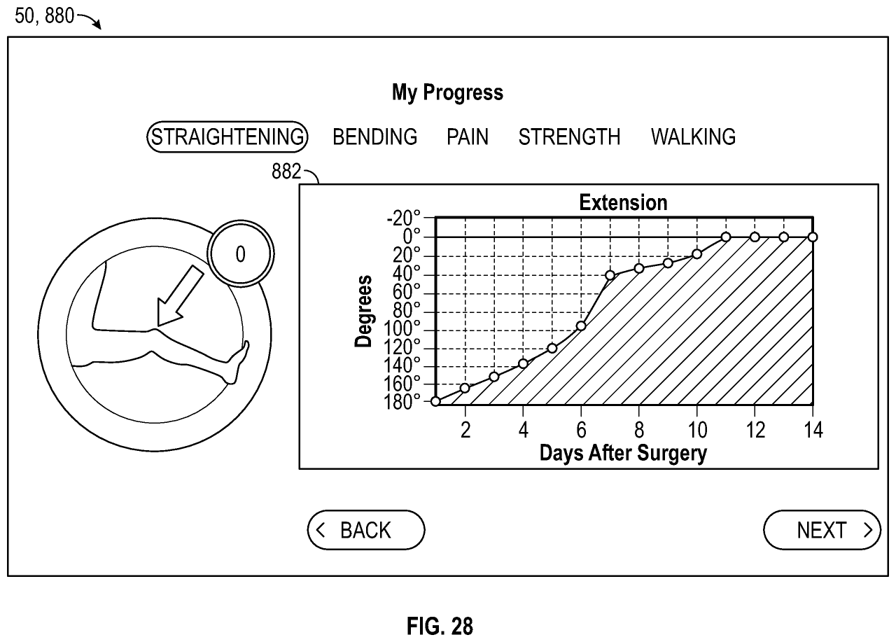

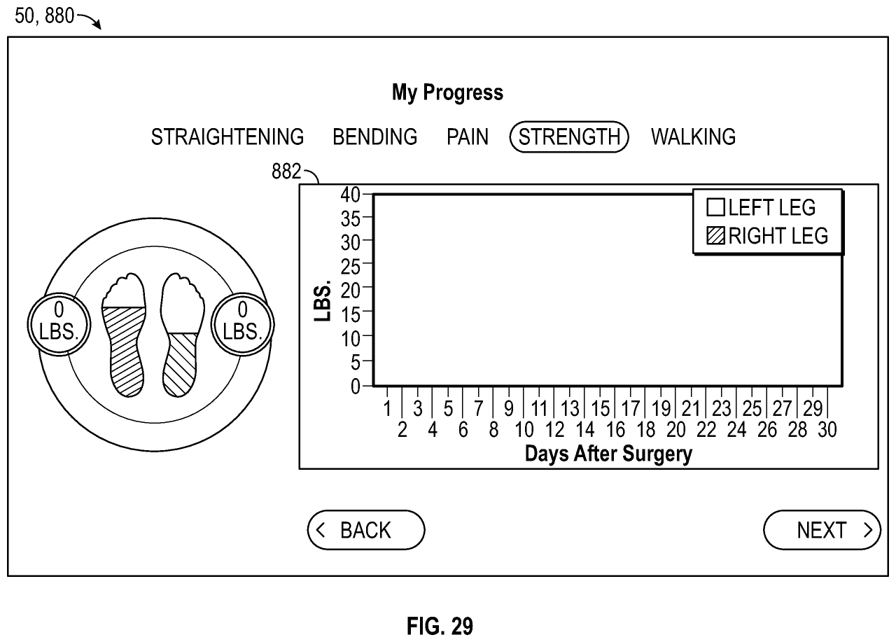

FIGS. 28-29 show two example progress data screens 880 of the patient interface 50. The first progress data screen 880 presents a progress graph 882 for each of several different parameters related to the treatment plan 154. For example, the progress graphs 882 may include historical data for straightening and bending of the knee pain, strength (lbs. pressure), and walking (steps per day). The progress graphs 882 may show identical data or data similar to what is presented on the treatment parameter graphs 136 of the clinician interface 20.

In some embodiments, a computer, such as the server 30, is configured to automatically modify the treatment plan 154 in response to satisfaction by the patient of a predetermined condition. For example, the treatment plan 154 may be limited in speed, velocity, or pressure settings or number of sessions per day until a predetermined condition is satisfied. In another example, the treatment plan 154 may include only certain types of session periods, such as passive type exercises, until the predetermined condition is satisfied. The predetermined condition may include, for example, a successful post-operative checkup; or completion of a predetermined number of sessions or satisfying a performance benchmark within the treatment plan. Such a benchmark may include, for example, walking X number of steps in a day, or some given RPM speed or a given number of pounds of force using the treatment apparatus 70. In some embodiments, the computer is configured to increase at least one of a frequency, a duration, or an intensity of an aspect of the treatment plan 154 in response to performance or occurrence of the predetermined condition. In some embodiments, the computer is configured to decrease at least one of a frequency, a duration, or an intensity of an aspect of the treatment plan 154 in response to a performance or occurrence of the condition. The predetermined condition may include, for example, the patient reporting pain in excess of a given value, or an inability to complete one or more activities within the treatment plan 154, or a sudden decrease in walking performed by the patient.

In some embodiments, the patient interface 50 may provide a prompt to the patient in response to occurrence of the predetermined condition. For example, in a session period where the patient is expected to maintain the stationary cycling machine at a speed of between 40 and 50 RPM, the predetermined condition may include the cycling machine operating below 30 RPM for a period of 5 seconds. In that case, the patient interface 50 may provide a prompt asking the patient if they are having trouble or pain in performing the activity. The prompts may narrow down a problem. For example, if the patient is unable to perform a given activity, then a computer, such as the server 30, may automatically modify the treatment plan 154 to include activities that are easier for the patient to complete, such as only passive or only assisted session periods. Alternatively, the treatment plan 154 may be suspended until the clinician or another qualified person, such as an orthopedic surgeon, directs the system 10 to re-enable the treatment plan 154. Additionally or alternatively, the patient's responses to the prompts may generate an alert to the clinician.

In some embodiments, the system may communicate an alert message to the clinician using a communication message, such as a pager message or a text message or an email. The alert message may include pseudonymized data and/or anonymized data or use any privacy enhancing technology to prevent confidential patient data from being communicated in a way that could violate patient confidentiality requirements. Such privacy enhancing technologies may enable compliance with laws, regulations, or other rules of governance such as, but not limited to, the Health Insurance Portability and Accountability Act (HIPAA), or the General Data Protection Regulation (GDPR), wherein the patient may be deemed a “data subject”. For example, an alert message may direct the clinician that a particular type of alert exists, such as a patient reporting wound splitting, without identifying which patient made the report. The alert message may direct the clinician to check the clinician interface 20 for more specific details regarding the alert.

The foregoing description, for purposes of explanation, use specific nomenclature to provide a thorough understanding of the described embodiments. However, it should be apparent to one skilled in the art that the specific details are not required to practice the described embodiments. Thus, the foregoing descriptions of specific embodiments are presented for purposes of illustration and description. They are not intended to be exhaustive or to limit the described embodiments to the precise forms disclosed. It should be apparent to one of ordinary skill in the art that many modifications and variations are possible in view of the above teachings.

The above discussion is meant to be illustrative of the principles and various embodiments. Numerous variations and modifications will become apparent to those skilled in the art once the above disclosure is fully appreciated. It is intended that the following claims be interpreted to embrace all such variations and modifications.

The various aspects, embodiments, implementations, or features of the described embodiments can be used separately or in any combination. The embodiments disclosed herein are modular in nature and can be used in conjunction with or coupled to other embodiments.

Any of the systems and methods described in this disclosure may be used in connection with rehabilitation. Rehabilitation may be directed at cardiac rehabilitation, rehabilitation from stroke, multiple sclerosis, Parkinson's disease, a brain injury, a spinal cord injury, a spinal cord disease, a joint injury, a joint disease, or the like. Rehabilitation can further involve muscular contraction improving blood flow and lymphatic flow, engaging the brain and nervous system to control and affect a traumatized area to increase the speed of healing, reversing or reducing pain, reversing or reducing stiffness, recovering range of motion, cardiovascular engagement to stimulate the release of pain blocking hormones and encourage freshly oxygenated blood flow to aid in an overall feeling of well-being. Rehabilitation may be provided for individuals of average height in reasonably good physical condition having no substantial deformities, as well as individuals more typically in need of rehabilitation, such as those that are elderly, obese, injured and/or have a severely limited range of motion. Unless expressly stated otherwise, is to be understood that rehabilitation includes prehabilitation (also referred to as “prehabilitation” or “prehab”). Prehabilitation may be used as a preventative procedure or as a pre-surgical or pre-treatment procedure. Prehabilitation may include any action performed by or on a patient (or directed to be performed by or on a patient, including, without limitation, remotely or distally through telemedicine) to, without limitation, prevent or reduce a likelihood of injury (e.g., prior to the occurrence of the injury); improve recovery time subsequent to surgery; improve strength subsequent to surgery; or any of the foregoing with respect to any non-surgical clinical treatment plan to be undertaken for the purpose of ameliorating or mitigating injury, dysfunction, or other negative consequence of surgical or non-surgical treatment on any external or internal part of a patient's body. For example, a mastectomy may require prehabilitation to strengthen muscles or muscle groups affected directly or indirectly by the mastectomy. As a further non-limiting example, the removal of an intestinal tumor, the repair of a hernia, open-heart surgery or other procedures performed on internal organs or structures, whether to repair those organs or structures, to excise them or parts of them, to treat them, etc., can require cutting through and harming numerous muscles and muscle groups in or about, without limitation, the abdomen, the ribs and/or the thoracic cavity. Prehabilitation can improve a patient's speed of recovery, measure of quality of life, level of pain, etc. in all the foregoing procedures. In one embodiment of prehabilitation, a pre-surgical procedure or a pre-non-surgical-treatment may include one or more sets of exercises for a patient to perform prior to such procedure or treatment. Performance of the one or more sets of exercises may be required in order to qualify for an elective surgery, such as a knee replacement. The patient may prepare an area of his or her body for the surgical procedure by performing the one or more sets of exercises, thereby strengthening muscle groups, improving existing muscle memory, reduce pain, reduce stiffness, establishing new muscle memory, enhancing mobility (i.e., improve range of motion), improving blood flow, and/or the like.

Consistent with the above disclosure, the examples of assemblies enumerated in the following clauses are specifically contemplated and are intended as a non-limiting set of examples.

1. A computer implemented system for managing a treatment plan, comprising: a clinician interface comprising a patient profile display and a protocol management display, the patient profile display presenting historical data regarding performance of the treatment plan by a patient, and the protocol management display presenting controls for modifying a treatment protocol within the treatment plan; wherein the treatment protocol comprises a plurality of sessions for treatment of a body part of the patient; wherein each session within the plurality of sessions comprises an ordered combination of session periods, with each of the session periods including a particular activity for treating the body part of the patient; wherein each of the session periods is selected from a group comprising a passive period, an assisted period, an active period, and a resistance period; wherein the particular activity of each passive period includes moving the body part by an external force; wherein the particular activity of each assisted period includes moving the body part by the patient with assistance of the external force; wherein the particular activity of each active period includes the patient moving the body part without assistance of the external force; and wherein the particular activity of each resistance period includes the patient actively moving the body part against a resistance force.

2. The computer implemented system of claim 1, wherein the treatment plan comprises a rehabilitation regimen, and the body part comprises at least one of a joint, a bone, or a muscle group.

3. The computer implemented system of claim 1, further comprising a computer configured to automatically modify the treatment plan in response to satisfaction by the patient of a predetermined condition.

4. The computer implemented system of claim 3, wherein the predetermined condition comprises the patient satisfying a performance benchmark within the treatment plan; and wherein the computer is configured to increase at least one of a frequency, a duration, or an intensity of an aspect of the treatment plan in response to performance of the predetermined condition.

5. The computer implemented system of claim 3, wherein the predetermined condition comprises the patient failing to satisfy a performance benchmark within the treatment protocol; and wherein the computer is configured to decrease at least one of a frequency, a duration, or an intensity of an aspect of the treatment protocol in response to performance of the predetermined condition.

6. The computer implemented system of claim 1, wherein the treatment protocol is one of a plurality of treatment protocols within the treatment plan; and further comprising the clinician interface including a plan modification control configured to modify the plurality of treatment protocols within the treatment plan.

7. A system for remote treatment, comprising: a clinician interface presenting a protocol management display presenting controls for modifying a treatment plan for treatment of a body part of a patient; a patient interface including an output device and an input device for communicating information to and from the patient; and a treatment apparatus configured to be manipulated by the patient for performing a rehabilitation regimen upon the body part, with the body part comprising at least one of a joint, a bone, or a muscle group; and wherein the patient interface and the treatment apparatus are each configured to operate from a patient location geographically separate from a location of the clinician interface.

8. The system of claim 7, wherein at least one of the patient interface or the treatment apparatus is configured to communicate with a remote computer to receive the treatment plan and to report data regarding performance by the patient in following the treatment plan.

9. The system of claim 7, wherein the treatment apparatus comprises an actuator configured to provide at least one of an external force or a resistance to the body part of the patient.

10. The system of claim 7, further comprising a force sensor configured to measure a force applied by the patient.

11. The system of claim 7, wherein the treatment apparatus comprises a position sensor configured to measure at least one of a linear motion or an angular motion of the body part of the patient.

12. The system of claim 7, wherein the clinician interface comprises an overview display including summary information regarding each of a plurality of different patients.

13. The system of claim 7, further comprising a server computer configured to store data regarding the treatment plan.

14. The system of claim 7, further comprising a server computer configured to store data regarding performance by the patient in following the treatment plan.

15. A clinician user interface generated by a computer and comprising: a profile display presenting information regarding a treatment history of a patient; a protocol management display presenting a treatment plan, with the treatment plan comprising a plurality of treatment protocols; a plan modification control configured to modify the plurality of treatment protocols of the treatment plan; wherein each treatment protocol of the plurality of treatment protocols comprises a plurality of sessions for treatment of a body part of the patient; wherein each session of the plurality of sessions comprises an ordered combination of session periods, with each of the session periods including a particular activity for treating the body part of the patient.

16. The clinician user interface of claim 15, wherein each of the session periods is selected from a group comprising a passive period, an assisted period, an active period, and a resistance period; wherein the particular activity of each passive period includes moving the body part by an external force; wherein the particular activity of each assisted period includes moving the body part by the patient with assistance of the external force; wherein the particular activity of each active period includes the patient moving the body part without assistance of the external force; and wherein the particular activity of each resistance period includes the patient actively moving the body part against a resistance force.

17. The clinician user interface of claim 15, further comprising a drag-and-drop interface for configuring the ordered combination of the session periods.

18. The clinician user interface of claim 15, wherein the information regarding the treatment history of the patient comprises a graphical representation of each of a plurality of different parameters over time.

19. The clinician user interface of claim 15, wherein the protocol management display includes a control for modifying a parameter of the treatment plan.

20. The clinician user interface of claim 19, wherein the control for modifying the parameter of the treatment plan is configured to modify at least one of a speed, a force, or a duration of the particular activity within a selected one of the session periods.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

The various aspects, embodiments, implementations, or features of the described embodiments can be used separately or in any combination. The embodiments disclosed herein are modular in nature and can be used in conjunction with or coupled to other embodiments.

Consistent with the above disclosure, the examples of assemblies enumerated in the following clauses are specifically contemplated and are intended as a non-limiting set of examples.

What is claimed is:

1. A computer implemented system for managing a treatment plan, comprising:
a clinician interface comprising a patient profile display and a protocol management display, the patient profile display presenting historical data regarding performance of the treatment plan by a patient, and the protocol management display presenting controls for modifying a treatment protocol within the treatment plan;
wherein the treatment protocol comprises a plurality of sessions for treatment of a body part of the patient;
wherein each session within the plurality of sessions comprises an ordered combination of session periods, with each of the session periods including a particular activity for treating the body part of the patient;
wherein each of the session periods is selected from a group comprising a passive period, an assisted period, an active period, and a resistance period;
wherein the particular activity of each passive period includes moving the body part by an external force;
wherein the particular activity of each assisted period includes moving the body part by the patient with assistance of the external force;
wherein the particular activity of each active period includes the patient moving the body part without assistance of the external force; and
wherein the particular activity of each resistance period includes the patient actively moving the body part against a resistance force.

2. The computer implemented system of claim 1, wherein the treatment plan comprises a rehabilitation regimen, and the body part comprises at least one of a joint, a bone, or a muscle group.

3. The computer implemented system of claim 1, further comprising a computer configured to automatically modify the treatment plan in response to satisfaction by the patient of a predetermined condition.

4. The computer implemented system of claim 3, wherein the predetermined condition comprises the patient satisfying a performance benchmark within the treatment plan; and
wherein the computer is configured to increase at least one of a frequency, a duration, or an intensity of an aspect of the treatment plan in response to performance of the predetermined condition.

5. The computer implemented system of claim 3, wherein the predetermined condition comprises the patient failing to satisfy a performance benchmark within the treatment protocol; and
wherein the computer is configured to decrease at least one of a frequency, a duration, or an intensity of an aspect of the treatment protocol in response to performance of the predetermined condition.

6. The computer implemented system of claim 1, wherein the treatment protocol is one of a plurality of treatment protocols within the treatment plan; and
further comprising the clinician interface including a plan modification control configured to modify the plurality of treatment protocols within the treatment plan.

7. A system for remote treatment, comprising:
a clinician interface presenting a protocol management display presenting controls for modifying a treatment plan for treatment of a body part of a patient;
a patient interface including an output device and an input device for communicating information to and from the patient; and
a treatment apparatus configured to be manipulated by the patient for performing a rehabilitation regimen upon the body part, with the body part comprising at least one of a joint, a bone, or a muscle group; and
wherein the patient interface and the treatment apparatus are each configured to operate from a patient location geographically separate from a location of the clinician interface.

8. The system of claim 7, wherein at least one of the patient interface or the treatment apparatus is configured to communicate with a remote computer to receive the treatment plan and to report data regarding performance by the patient in following the treatment plan.

9. The system of claim 7, wherein the treatment apparatus comprises an actuator configured to provide at least one of an external force or a resistance to the body part of the patient.

10. The system of claim 7, further comprising a force sensor configured to measure a force applied by the patient.

11. The system of claim 7, wherein the treatment apparatus comprises a position sensor configured to measure at least one of a linear motion or an angular motion of the body part of the patient.

12. The system of claim 7, wherein the clinician interface comprises an overview display including summary information regarding each of a plurality of different patients.

13. The system of claim 7, further comprising a server computer configured to store data regarding the treatment plan.

14. The system of claim 7, further comprising a server computer configured to store data regarding performance by the patient in following the treatment plan.

15. A clinician user interface generated by a computer and comprising:
a profile display presenting information regarding a treatment history of a patient;
a protocol management display presenting a treatment plan, with the treatment plan comprising a plurality of treatment protocols;
a plan modification control configured to modify the plurality of treatment protocols of the treatment plan;
wherein each treatment protocol of the plurality of treatment protocols comprises a plurality of sessions for treatment of a body part of the patient;
wherein each session of the plurality of sessions comprises an ordered combination of session periods, with each of the session periods including a particular activity for treating the body part of the patient.

16. The clinician user interface of claim 15,
wherein each of the session periods is selected from a group comprising a passive period, an assisted period, an active period, and a resistance period;
wherein the particular activity of each passive period includes moving the body part by an external force;
wherein the particular activity of each assisted period includes moving the body part by the patient with assistance of the external force;
wherein the particular activity of each active period includes the patient moving the body part without assistance of the external force; and
wherein the particular activity of each resistance period includes the patient actively moving the body part against a resistance force.

17. The clinician user interface of claim 15, further comprising a drag-and-drop interface for configuring the ordered combination of the session periods.

18. The clinician user interface of claim 15, wherein the information regarding the treatment history of the patient comprises a graphical representation of each of a plurality of different parameters over time.

19. The clinician user interface of claim 15, wherein the protocol management display includes a control for modifying a parameter of the treatment plan.

20. The clinician user interface of claim 19, wherein the control for modifying the parameter of the treatment plan is configured to modify at least one of a speed, a force, or a duration of the particular activity within a selected one of the session periods.
